(19) United States
(12) Patent Application Publication
(10) Pub. No.: US 2008/0255106 A1
(43) Pub. Date: Oct. 16, 2008

(54) Novel 2-Phenyl-Imidazo[4,5-B]Pyridine Derivatives as Inhibitors of Glycogen Synthase Kinase for the Treatment of Dementia and Neurodegenerative Disorders

(75) Inventors: Per I. Arvidsson, Sodertalje (SE); Erwan Arzel, Sodertalje (SE); Jeremy Burrows, Sodertalje (SE); Helena Gyback, Sodertalje (SE); Tobias Rein, Sodertalje (SE); Didier Rotticci, Sodertalje (SE); Peter Soderman, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/088,897

(22) PCT Filed: Oct. 2, 2006
(86) PCT No.: PCT/SE06/01115

§ 371 Date: Apr. 1, 2008

(30) Foreign Application Priority Data
Oct. 3, 2005 (SE) 0502173-8

(51) Int. Cl. A61K 31/437; A61P 25/00; C07D 471/04; A61K 31/541; A61K 31/496; A61P 3/10; A61K 31/5377

(52) U.S. Cl. 514/228.5; 546/118; 544/127; 544/58.2; 544/362; 544/121; 514/234.2; 514/303; 514/253.04

(57) ABSTRACT

Compounds of formula I

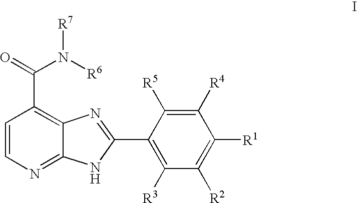

wherein R¹, R², R³, R⁴, R⁵, R⁶ and R⁷ are as defined in the specification, as a base or a pharmaceutically acceptable salt, solvate or solvate of salt thereof, processes for their preparation and new intermediates used therein, pharmaceutical formulations containing said compounds and to the use of said compounds in therapy.

### TECHNICAL FIELD OF INVENTION

The present invention relates to new compounds of formula I, as a free base or a pharmaceutically acceptable salt, solvate or solvate of salt thereof, to pharmaceutical formulations containing said compounds and to the use of said compounds in therapy. The present invention further relates to a process for the preparation of compounds of formula I and to new intermediates used therein.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

### Alzheimer's Disease (AD) Dementias, and Taupathies

AD is characterized by cognitive decline, cholinergic dysfunction and neuronal death, neurofibrillary tangles and senile plaques consisting of amyloid-β deposits. The sequence of these events in AD is unclear, but they are believed to be related. Glycogen synthase kinase 3β (GSK3β) or Tau (τ) phosphorylating kinase selectively phosphorylates the microtubule associated protein r in neurons at sites that are hyperphosphorylated in AD brains. Hyperphosphorylated protein r has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of τ and a paired helical filaments-like state via induction of GSK3β activity, followed by disruption of axonal transport and neuronal death (Imahori and Uchida., J. Biochem 121:179-188, 1997). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 93:2719-2723, 1996). Acetyl-Co-A is critical for the synthesis of acetylcholine, a neurotransmitter with cognitive functions. Thus, GSK3β inhibition may have beneficial effects in progression as well as the cognitive deficits associated with Alzheimer's disease and other above-referred to diseases.

### Chronic and Acute Neurodegenerative Diseases

Growth factor mediated activation of the PI3K/Akt pathway has been shown to play a key role in neuronal survival. The activation of this pathway results in GSK3β inhibition. Recent studies (Bhat et. al., PNAS 97:11074-11079 (2000)) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as Alzheimer's Disease, Parkinson's Disease, amyotrophic lateral sclerosis, Huntington's Disease and HIV dementia, ischemic stroke and head trauma. Lithium was neuroprotective in inhibiting apoptosis in cells and in the brain at doses that resulted in the inhibition of GSK3β. Thus GSK3β inhibitors could be useful in attenuating the course of neurodegenerative diseases.

### Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The recent discovery that lithium inhibits GSK3 at therapeutic concentrations has raised the possibility that this enzyme represents a key target of lithium's action in the brain (Stambolic et al., Curr. Biol. 6:1664-1668, 1996; Klein and Melton; PNAS 93:8455-8459, 1996). Inhibition of GSK3β may therefore be of therapeutic relevance in the treatment of BD as well as in AD patients that have affective disorders.

### Schizophrenia

GSK3 is involved in signal transduction cascades of multiple cellular processes, particularly during neural development. Kozlovsky et al (Am J Psychiatry 2000 May; 157(5):831-3) found that GSK3β levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 9:1379-1383 (1998)).

### Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February; 49(2):263-71). Inhibition of GSK3 increases the activity of glycogen synthase thereby decreasing glucose levels by its conversion to glycogen. GSK3 inhibition may therefore be of therapeutic relevance in the treatment of Type I and Type II diabetes and diabetic neuropathy.

### Hair Loss

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell 1998 Nov. 25; 95 (5):605-14)). The new follicles formed sebaceous glands and dermal papilla, normally established only in embryogenesis. Thus GSK3 inhibition may offer treatment for baldness.

### Oral Contraceptives

Vijajaraghavan et al. (Biol Reprod 2000 June; 62 (6):1647-54) reported that GSK3 is high in motile versus immotile sperm. Immunocytochemistry revealed that GSK3 is present in the flagellum and the anterior portion of the sperm head. These data suggest that GSK3 could be a key element underlying motility initiation in the epididymis and regulation of mature sperm function. Inhibitors of GSK3 could be useful as contraceptives for males.

### Bone-Related Disorders

It has been shown that GSK3 inhibitors could be used for treatment of bone-related disorders. This has been discussed in e.g. Tobias et al., Expert Opinion on Therapeutic Targets, February 2002, pp 41-56.

### DISCLOSURE OF THE INVENTION

The object of the present invention is to provide compounds having a selective inhibiting effect at GSK3 as well as having a good bioavailability. Accordingly, the present invention provides a compound of the formula I:

The present invention relates to a compound of formula I:

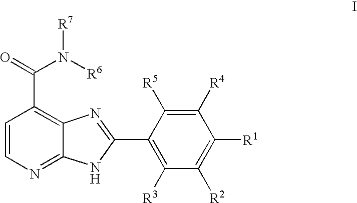

wherein;
R¹ is selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ are independently selected from hydrogen, C₁₋₃alkyl and C₁₋₃haloalkyl;
R⁶ and R⁷ are independently selected from hydrogen, C₁₋₆alkyl, C₁₋₆alkylaryl, aryl and heteroaryl, said C₁₋₆alkyl, C₁₋₆alkylaryl, aryl and heteroaryl are optionally substituted with one or more A; or
R⁶ and R⁷ may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more R^{j} or A.
R^{a} is hydrogen, C₁₋₃alkyl or C₁₋₃haloalkyl, said C₁₋₃alkyl or C₁₋₃haloalkyl is optionally substituted with one or more C₁₋₃alkoxy;
R^{b} and R^{c} are independently selected from hydrogen, C₁₋₆alkyl and C₁₋₆haloalkyl, said C₁₋₆alkyl or C₁₋₆haloalkyl is optionally substituted with one or more OR^{a} or NR^{d}R^{e} or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃alkoxy and in which any sulphur atom is optionally oxidised to —SO₂—;
R^{d} and R^{e} are independently selected from hydrogen, C₁₋₆alkyl and C₁₋₆haloalkyl, said C₁₋₆alkyl or C₁₋₆haloalkyl is optionally substituted with one or more OR^{a}; or
R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is hydrogen, C₁₋₃alkyl or C₁₋₃haloalkyl, said C₁₋₃alkyl or C₁₋₃haloalkyl is optionally substituted with one or more C₁₋₃alkoxy;
Rⁱ is C₁₋₃alkyl or C₁₋₃haloalkyl, said C₁₋₃alkyl or C₁₋₃haloalkyl is optionally substituted with one or more OR^{a};
R^{j} is aryl or heteroaryl, wherein said aryl or heteroaryl is optionally substituted with one or more C₁₋₃alkyl, OR^{a}, halo or CN;
A is halo, CN, OR^{a}, or NR^{b}R^{c};
as a free base or a pharmaceutically acceptable salt, solvate or solvate of a salt thereof.

The present invention also relates to a compound of the formula I:

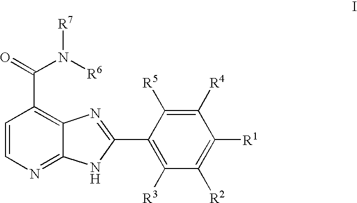

wherein;
R¹ is hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ or C(O)R^{j};
R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ are independently selected from hydrogen, C₁₋₃alkyl and C₁₋₃haloalkyl;
R⁶ and R⁷ are independently selected from hydrogen, C₁₋₆alkyl and heteroaryl, said C₁₋₆alkyl and heteroaryl optionally substituted with one or more A;
R^{a} is hydrogen, C₁₋₃alkyl or C₁₋₃haloalkyl, said C₁₋₃alkyl or C₁₋₃haloalkyl optionally substituted with one or more C₁₋₃alkoxy;
R^{b} and R^{c} are independently selected from hydrogen, C₁₋₆alkyl and C₁₋₆haloalkyl, said C₁₋₆alkyl or C₁₋₆haloalkyl optionally substituted with one or more OR^{a} or NR^{d}R^{e} or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{d} and R^{e} are independently selected from hydrogen, C₁₋₆alkyl and C₁₋₆haloalkyl, said C₁₋₆alkyl or C₁₋₆haloalkyl optionally substituted with one or more OR^{a}; or
R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is hydrogen, C₁₋₃alkyl or C₁₋₃haloalkyl, said C₁₋₃alkyl or C₁₋₃haloalkyl optionally substituted with one or more C₁₋₃alkoxy;
Rⁱ is C₁₋₃alkyl or C₁₋₃haloalkyl, said C₁₋₃alkyl or C₁₋₃haloalkyl optionally substituted with one or more OR^{a};
R^{j} is aryl or heteroaryl, wherein said aryl or heteroaryl is optionally substituted with one or more C₁₋₃alkyl, OR^{a}, halo or CN;
A is halo, CN, OR^{a}, or NR^{b}R^{c};
as a free base or a pharmaceutically acceptable salt, solvate or solvate of a salt thereof.

One embodiment of the present invention provides a compound of the formula I, wherein

R¹ is hydrogen, C₁₋₃haloalkyl, SO₂NR^{b}R^{c}, C(O)NR^{b}R^{c}, CH₂NR^{b}R^{c}, CH₂OR^{h}, or SO₂Rⁱ;
R² and R⁴ are independently selected from hydrogen, halo, CN, NO₂, C₁₋₃alkyl, C₁₋₃haloalkyl, OR^{a}, C(O)NR^{b}R^{c}, CH₂NR^{b}R^{c}, CH₂OR^{h}, and SO₂Rⁱ;
R³ and R⁵ are independently selected from hydrogen and C₁₋₃haloalkyl;
R⁶ and R⁷ are independently selected from hydrogen, C₁₋₆alkyl and heteroaryl, said C₁₋₆alkyl and heteroaryl optionally substituted with one or more A;
R^{a} is C₁₋₃alkyl or C₁₋₃haloalkyl;
R^{b} and R^{c} are independently selected from C₁₋₆alkyl and C₁₋₆haloalkyl; or
R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is C₁₋₃alkyl or C₁₋₃haloalkyl;
Rⁱ is C₁₋₃alkyl or C₁₋₃haloalkyl;
A is halo, CN, OR^{a}, or NR^{b}R^{c};
as a free base or a pharmaceutically acceptable salt, solvate or solvate of a salt thereof.

Another embodiment of the present invention provides a compound of the formula I, wherein

R¹ is hydrogen, C₁₋₃haloalkyl, SO₂NR^{b}R^{c}, C(O)NR^{b}R^{c}, CH₂NR^{b}R^{c}, or SO₂Rⁱ;
R² and R⁴ are independently selected from hydrogen, C₁₋₃haloalkyl, CH₂OR^{h}, and SO₂Rⁱ;
R³ and R⁵ are independently selected from hydrogen and C₁₋₃haloalkyl;
R⁶ and R⁷ are independently selected from hydrogen and C₁₋₆alkyl, said C₁₋₆alkyl optionally substituted with one or more A;
R^{a} is C₁₋₃alkyl;
R^{b} and R^{c} are independently C₁₋₆alkyl; or
R^{b} and R^{c} may, together with the atom to which they are attached, form a 6-membered heterocyclic ring containing one or more heteroatoms selected from N or O, wherein said heterocyclic ring is optionally substituted with one a C₁₋₃alkyl;
R^{h} is C₁₋₃haloalkyl;
Rⁱ is C₁₋₃alkyl;

### A is OR^{a};

as a free base or a pharmaceutically acceptable salt, solvate or solvate of a salt thereof.

Yet another embodiment of the present invention provides a compound of the formula I, wherein

R¹ is selected from hydrogen, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R² and R⁴ are independently selected from hydrogen, halo, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ are independently selected from hydrogen, C₁₋₃alkyl and C₁₋₃haloalkyl;
R⁶ and R⁷ are independently selected from hydrogen, C₁₋₆alkyl, C₁₋₆alkylaryl, aryl and heteroaryl, said C₁₋₆alkyl, C₁₋₆alkylaryl, aryl and heteroaryl are optionally substituted with one or more A; or
R⁶ and R⁷ may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more R^{j} or A.
R^{a} is hydrogen, C₁₋₃alkyl or C₁₋₃haloalkyl, said C₁₋₃alkyl or C₁₋₃haloalkyl is optionally substituted with one or more C₁₋₃alkoxy;
R^{b} and R^{c} are independently selected from hydrogen, C₁₋₆alkyl and C₁₋₆haloalkyl, said C₁₋₆alkyl or C₁₋₆haloalkyl is optionally substituted with one or more OR^{a} or NR^{d}R^{e} or
R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃alkoxy and in which any sulphur atom is optionally oxidised to —SO₂—;
R^{d} and R^{e} are independently selected from hydrogen, C₁₋₆alkyl and C₁₋₆haloalkyl, said C₁₋₆alkyl or C₁₋₆haloalkyl is optionally substituted with one or more OR^{a}; or
R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is hydrogen, C₁₋₃alkyl or C₁₋₃haloalkyl, said C₁₋₃alkyl or C₁₋₃haloalkyl is optionally substituted with one or more C₁₋₃alkoxy;
Rⁱ is C₁₋₃alkyl or C₁₋₃haloalkyl, said C₁₋₃alkyl or C₁₋₃haloalkyl is optionally substituted with one or more OR^{a};
R^{j} is aryl or heteroaryl, wherein said aryl or heteroaryl is optionally substituted with one or more C₁₋₃alkyl, OR^{a}, halo or CN;
A is halo, CN, OR^{a}, or NR^{b}R^{c};
as a free base or a pharmaceutically acceptable salt, solvate or solvate of a salt thereof.

Another embodiment of the present invention relates to a compound of the formula I, wherein

R¹ is selected from hydrogen, C₁₋₃haloalkyl, C(O)NR^{b}R^{c}, CH₂NR^{b}R^{c}, SO₂Rⁱ, and SO₂R^{b}R^{c};
R² and R⁴ are independently selected from hydrogen, halo, C₁₋₃haloalkyl, OR^{a}, CH₂NR^{b}R^{c}, CH₂OR^{h} and SO₂Rⁱ;
R³ and R⁵ are independently selected from hydrogen or C₁₋₃haloalkyl;
R⁶ and R⁷ are independently selected from hydrogen, C₁₋₆alkyl, C₁₋₆alkylaryl, aryl and heteroaryl, said C₁₋₆alkyl, C₁₋₆alkylaryl, aryl and heteroaryl are optionally substituted with one or more A; or
R⁶ and R⁷ may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more R^{j} or A.
R^{a} is C₁₋₃alkyl;
R^{b} and R^{c} are independently selected from hydrogen, C₁₋₆alkyl and C₁₋₆haloalkyl or
R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃alkoxy and in which any sulphur atom is optionally oxidised to —SO₂—;
R^{h} is C₁₋₃haloalkyl,
Rⁱ is C₁₋₃alkyl;
R^{j} is aryl or heteroaryl, wherein said aryl or heteroaryl is optionally substituted with one or more C₁₋₃alkyl, OR^{a}, halo or CN;
A is halo, CN or OR^{a},
as a free base or a pharmaceutically acceptable salt, solvate or solvate of a salt thereof.

According to one embodiment of the present invention, R² and R³ are hydrogen.

A further embodiment of the present invention provides a compound of the formula I, wherein

R¹ is selected from hydrogen, C₁₋₃haloalkyl, C(O)NR^{b}R^{c}, CH₂NR^{b}R^{c}, SO₂Rⁱ and SO₂R^{b}R^{c};
R² and R⁴ are independently selected from hydrogen, halo, C₁₋₃haloalkyl, OR^{a}, CH₂NR^{b}R^{c}, CH₂OR^{h} and SO₂Rⁱ;
R⁶ and R⁷ are independently selected from hydrogen, C₁₋₆alkyl, C₁₋₆alkylaryl, aryl and heteroaryl, said C₁₋₆alkyl, C₁₋₆alkylaryl, aryl and heteroaryl are optionally substituted with one or more A; or
R⁶ and R⁷ may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more R^{j} or A.
R^{a} is C₁₋₃alkyl;
R^{b} and R^{c} are independently selected from hydrogen, C₁₋₆alkyl and C₁₋₆haloalkyl or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃ alkoxy and in which any sulphur atom is optionally oxidised to —SO₂—;
R^{h} is C₁₋₃haloalkyl;
Rⁱ is C₁₋₃alkyl;
R^{j} is aryl or heteroaryl, wherein said aryl or heteroaryl is optionally substituted with one or more C₁₋₃alkyl, OR^{a}, halo or CN;
A is halo, CN or OR^{a},
as a free base or a pharmaceutically acceptable salt, solvate or solvate of a salt thereof.

Another embodiment of the present invention relates to a compound of the formula I, wherein R⁶ and R⁷ are independently selected from hydrogen and C₁₋₆alkyl, said C₁₋₆alkyl is substituted with one OR^{a} and R^{a} is C₁₋₃alkyl. According to yet another embodiment of the present invention, said C₁₋₆alkyl is propyl and R^{a} is methyl.

According to a further embodiment of the present invention, said C₁₋₆alkylalkyl in R⁶ or R⁷ is C₁₋₃alkylaryl. According to yet another embodiment said C₁₋₃alkylaryl is methylphenyl.

The present invention also relates to compounds selected from:
N-(3-Methoxypropyl)-2-[2-(trifluoromethyl)phenyl]-3H-imidazo[4,5-b]pyridine-7-carboxamide hydrochloride;
N-(3-Methoxypropyl)-2-[3-(trifluoromethyl)phenyl]-3H-imidazo[4,5-b]pyridine-7-carboxamide hydrochloride;
N-(3-Methoxypropyl)-2-[4-(trifluoromethyl)phenyl]-3H-imidazo[4,5-b]pyridine-7-carboxamide hydrochloride;
N-(3-Methoxypropyl)-2-{3-[(2,2,3,3-tetrafluoropropoxy)methyl]phenyl}-3H-imidazo[4,5-b]pyridine-7-carboxamide hydrochloride;
N-(3-Methoxypropyl)-2-[4-(morpholin-4-ylmethyl)phenyl]-3H-imidazo[4,5-b]pyridine-7-carboxamide hydrochloride;
N-(3-Methoxypropyl)-2-{4-[(4-methylpiperazin-1-yl)carbonyl]phenyl}-3H-imidazo[4,5-b]pyridine-7-carboxamide hydrochloride;
N-(3-Methoxypropyl)-2-[4-(morpholin-4-ylcarbonyl)phenyl]-3H-imidazo[4,5-b]pyridine-7-carboxamide hydrochloride;
2-[3-Fluoro-4-(morpholin-4-ylmethyl)phenyl]-N-(3-methoxypropyl)-3H-imidazo[4,5-b]pyridine-7-carboxamide hydrochloride;
2-[3-Methoxy-4-(morpholin-4-ylmethyl)phenyl]-N-(3-methoxypropyl)-3H-imidazo[4,5-b]pyridine-7-carboxamide hydrochloride;
N-(3-Methoxypropyl)-2-[4-(morpholin-4-ylmethyl)-3-(trifluoromethyl)phenyl]-3H-imidazo[4,5-b]pyridine-7-carboxamide hydrochloride;
N-(3-Methoxypropyl)-2-[4-(pyrrolidin-1-ylsulfonyl)phenyl]-3H-imidazo[4,5-b]pyridine-7-carboxamide hydrochloride;
N-(3-Methoxypropyl)-2-{4-[(4-methylpiperazin-1-yl)sulfonyl]phenyl}-3H-imidazo[4,5-b]pyridine-7-carboxamide hydrochloride;
2-{4-[(1,1-Dioxidothiomorpholin-4-yl)methyl]phenyl}-N-(3-methoxypropyl)-3H-imidazo[4,5-b]pyridine-7-carboxamide hydrochloride;
N-(3-Methoxypropyl)-2-[4-(piperidin-1-ylmethyl)phenyl]-3H-imidazo[4,5-b]pyridine-7-carboxamide hydrochloride;
N-(3-Methoxypropyl)-2-[3-(morpholin-4-ylmethyl)phenyl]-3H-imidazo[4,5-b]pyridine-7-carboxamide hydrochloride;
N-(3-Methoxypropyl)-2-{3-[(4-methylpiperazin-1-yl)methyl]phenyl}-3H-imidazo[4,5-b]pyridine-7-carboxamide hydrochloride;
2-{4-[(Dipropylamino)sulfonyl]phenyl}-N-(3-methoxypropyl)-3H-imidazo[4,5-b]pyridine-7-carboxamide hydrochloride;
N-(3-Methoxypropyl)-2-[4-(methylsulfonyl)phenyl]-3H-imidazo[4,5-b]pyridine-7-carboxamide hydrochloride;
N-(3-Methoxypropyl)-2-[3-(methylsulfonyl)phenyl]-3H-imidazo[4,5-b]pyridine-7-carboxamide hydrochloride;
2-[4-(Morpholin-4-ylmethyl)phenyl]-N-pyridin-3-yl-3H-imidazo[4,5-b]pyridine-7-carboxamide;
N-Cyclopentyl-g-[4-(morpholin-4-ylmethyl)phenyl]-2,7,9-triazabicyclo[4.3.0]nona-1,3,5,7-tetraene-5-carboxamide;
N-(3-Methoxybenzyl)-2-[4-(morpholin-4-ylmethyl)phenyl]-3H-imidazo[4,5-b]pyridine-7-carboxamide; and
3-[4-({2-[4-(Morpholin-4-ylmethyl)phenyl]-3H-imidazo[4,5-b]pyridin-7-yl}carbonyl)piperazin-1-yl]propanenitrile;
   as a free base or a pharmaceutically acceptable salt, solvate or solvate of a salt thereof.

The present invention also relates to a compound selected from:
7-Chloro-2-[2-(trifluoromethyl)phenyl]-3H-imidazo[4,5-b]pyridine;
7-Chloro-2-[3-(trifluoromethyl)phenyl]-3H-imidazo[4,5-b]pyridine;
7-Chloro-2-[4-(trifluoromethyl)phenyl]-3H-imidazo[4,5-b]pyridine;
7-Chloro-2-{3-[(2,2,3,3-tetrafluoropropoxy)methyl]phenyl}-3H-imidazo[4,5-b]pyridine;
Methyl 4-(3H-imidazo[4,5-h]pyridin-2-yl)benzoate;
7-Iodo-2-[4-(morpholin-4-ylmethyl)phenyl]-3H-imidazo[4,5-h]pyridine;
Methyl 4-(7-chloro-3H-imidazo[4,5-b]pyridin-2-yl)benzoate;
4-(7-Chloro-3H-imidazo[4,5-b]pyridin-2-yl)benzoic acid;
7-Chloro-2-[4-(morpholin-4-ylcarbonyl)phenyl]-3H-imidazo[4,5-b]pyridine;
7-Chloro-2-[4-(morpholin-4-ylmethyl)phenyl]-3H-imidazo[4,5-b]pyridine;
7-Chloro-2-[3-fluoro-4-(morpholin-4-ylmethyl)phenyl]-3H-imidazo[4,5-b]pyridine;
7-Chloro-2-[3-methoxy-4-(morpholin-4-ylmethyl)phenyl]-3H-imidazo[4,5-b]pyridine;
7-Chloro-2-[4-(morpholin-4-ylmethyl)-3-(trifluoromethyl)phenyl]-3H-imidazo[4,5-b]pyridine;
7-Chloro-2-[4-(pyrrolidin-1-ylsulfonyl)phenyl]-3H-imidazo[4,5-b]pyridine;
7-Chloro-2-{4-[(4-methylpiperazin-1-yl)sulfonyl]phenyl}-3H-imidazo[4,5-b]pyridine;
7-Chloro-2-{4-[(1,1-dioxidothiomorpholin-4-yl)methyl]phenyl}-3H-imidazo[4,5-b]pyridine;
7-Chloro-2-[4-(piperidin-1-ylmethyl)phenyl]-3H-imidazo[4,5-b]pyridine;
7-Chloro-2-[3-(morpholin-4-ylmethyl)phenyl]-3H-imidazo[4,5-b]pyridine;
7-Chloro-2-{3-[(4-methylpiperazin-1-yl)methyl]phenyl}-3H-imidazo[4,5-b]pyridine;
4-(7-Chloro-3H-imidazo[4,5-b]pyridin-2-yl)-N,N-dipropylbenzenesulfonamide;
7-Chloro-2-[4-(methylsulfonyl)phenyl]-3H-imidazo[4,5-b]pyridine;
7-Chloro-2-[3-(methylsulfonyl)phenyl]-3H-imidazo[4,5-b]pyridine; and
Methyl 8-[4-(morpholin-4-ylmethyl)phenyl]-2,7,9-triazabicyclo[4.3.0]nona-1,3,5,7-tetraene-5-carboxylate.

These compounds following compounds are useful as intermediates in the preparation of compounds according to Formula I:

Listed below are definitions of various terms used in the specification and claims to describe the present invention.

In this specification the term “alkyl” includes both straight and branched chain as well as cyclic alkyl groups. The term C₁₋₃alkyl having 1 to 3 carbon atoms and may be, but is not limited to, methyl, ethyl, n-propyl, i-propyl, or cyclopropyl. The term C₁₋₆alkyl having 1 to 6 carbon atoms and may be, but is not limited to, methyl, ethyl, n-propyl, i-propyl, n-butyl, i-butyl, s-butyl, t-butyl, n-pentyl, i-pentyl, t-pentyl, neo-pentyl, n-hexyl, i-hexyl, cyclopentyl or cyclohexyl.

The term “C₁₋₃alkoxy” includes both straight and branched chains. The term “C₁₋₃alkoxy” having 1 to 3 carbon atoms and may be, but is not limited to, methoxy, ethoxy, n-propoxy, or i-propoxy.

The term “halo” or “halogen” refers to fluorine, chlorine, bromine and iodine.

The term “haloalkyl” refers to an alkyl group, defined as above, in which one or several of the hydrogen substituents have been replaced by halogen substituents, in which the term halogen is defined as above.

The term “aryl” refers to an optionally substituted monocyclic or bicyclic hydrocarbon ring system containing at least one unsaturated aromatic ring. The “aryl” may be fused with a C₅₋₇cycloalkyl ring to form a bicyclic hydrocarbon ring system. Examples and suitable values of the term “aryl”, but not limiting, are phenyl, naphthyl, indanyl or tetralinyl.

The term “C₁₋₆alkylaryl”, includes both substituted and unsubstituted alkylaryl groups, which may be substituted on the alkyl and/or the aryl and may be, but are not limited to benzyl, methylphenyl or ethylphenyl.

As used herein, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, fluorenonyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl or heteroaromatic group has 1 to about 4, 1 to about 3, or 1 to 2 heteroatoms. In some embodiments, the heteroaryl or heteroaromatic group has 1 heteroatom.

The term “4-, 5-, 6- or 7-membered heterocyclic ring containing one or more heteroatoms independently selected from N, O, or S” refers to a mono- or bicyclic-heterocyclic ring which may be saturated or partly saturated and which may optionally contain a carbonyl function and which may be, but is not limited to, azetidinyl, imidazolidinyl, imidazolinyl, morpholinyl, piperazinyl, piperidinyl, piperidonyl, pyrazolidinyl, pyrazolinyl, pyrrolidinyl, pyrrolinyl, 1-methyl-1,4-diazepane, tetrahydropyranyl or thiomorpholinyl. In the case where the heterocyclic ring contains a heteroatom selected from S or N, these atoms may optionally be in an oxidised form such as SO or SO₂.

The term “hydrochloride” includes monohydrochloride, dihydrochloride, trihydrochloride and tetrahydrochloride salts.

A suitable pharmaceutically acceptable salt of the compound of the invention is, for example, an acid-addition salt, for example an inorganic or organic acid. In addition a suitable pharmaceutically acceptable salt of the compounds of the invention is an alkali metal salt, an alkaline earth metal salt or a salt with an organic base that affords a physiologically-acceptable cation.

Some compounds of formula I may have stereogenic centres and/or geometric isomeric centres (E- and Z-isomers), and it is to be understood that the invention encompasses all such optical, diastereoisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

An object of the invention is to provide compounds of formula I for therapeutic use, especially compounds that are useful for the prevention and/or treatment of conditions associated with glycogen synthase kinase-3 (GSK3) in mammals including man. Particularly, compounds of formula I exhibiting a selective affinity for GSK-3.

### Methods of Preparation

Another aspect of the present invention provides a process for preparing a compound of formula I as a free base or a pharmaceutically acceptable salt thereof. Throughout the following description of such processes it is understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Greene, P. G. M. Wuts, Wiley-Interscience, New York, 1999.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

### Methods of Preparation of Intermediates

The processes for the preparation of the intermediates, wherein R¹, R², R³, R⁴, R⁵, R⁶, R⁷, R^{b} and R^{c} are, unless specified otherwise, defined as in formula I, comprise of the following:

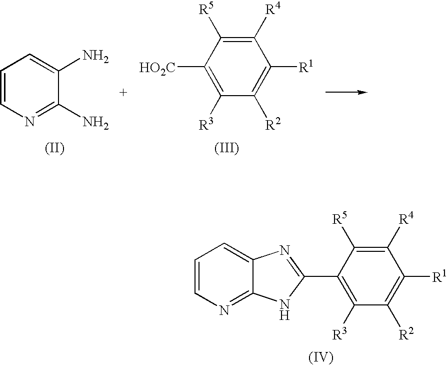

(i) Condensation of diamine II with a carboxylic acid of type III to give an intermediate IV can be performed by
(a) First, reacting II and III in the presence of a suitable catalyst, e.g. o-benzotriazol-1-yl-N,N,N′,N′-tetramethyluroniumhexafluorophosphate or O-(7-azabenzotriazol-1-yl)-N,N,N′,N′-tetramethyluronium hexafluorophosphate, in a solvent such as acetonitrile, dimethyl formamide, or a mixture thereof. A suitable base such as N,N-diisopropylethylamine may be used in the reaction, which can be performed at a temperature in the range of 0° C. to +20° C.
(b) Second, heating the resulting intermediate in a suitable organic acid such as acetic acid, at a temperature in the range of +150° C. to +200° C. using an oil bath or a microwave oven.

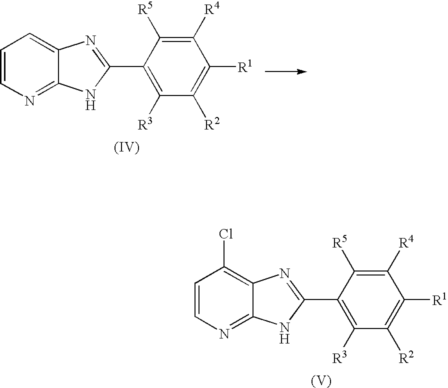

(ii) Conversion of a compound of type IV into a chloride of type V can be achieved by (a) first, reacting IV with an appropriate oxidant, e.g. m-chloroperbenzoic acid, in a suitable solvent, e.g. acetic acid, at a temperature in the range of +20° C. to +30° C.; (b) second, reaction of the formed intermediate with neat phosphorus oxychloride at a temperature in the range of +100° C. to +150° C. using an oil bath or a microwave oven.

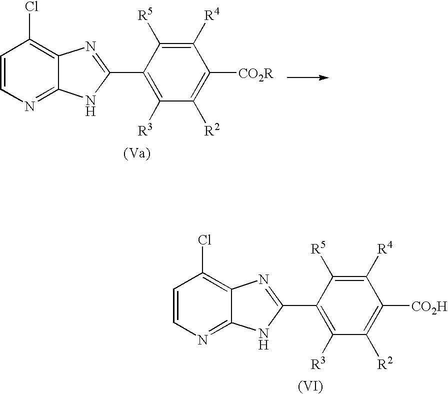

(iii) Hydrolysis of an ester of type Va (V, wherein R¹ is CO₂R and wherein R is methyl) to the corresponding acid VI might be effected by reaction with a suitable base, such as lithium, sodium or potassium hydroxide, or potassium carbonate, in mixtures of water and a suitable co-solvent, e.g. tetrahydrofuran or methanol, at a temperature in the range of +20° C. to +120° C. using an oil bath or a microwave oven.

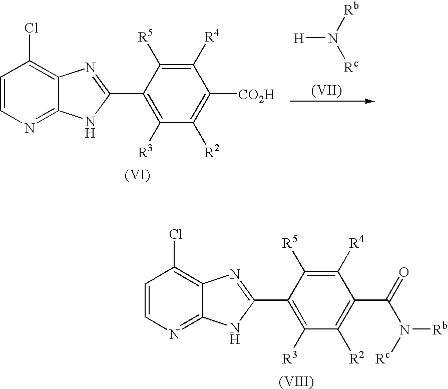

(iv) Formation of an amide of type VIII from the corresponding acid VI and an amine VII can be performed by reacting VI and VII in the presence of a suitable catalyst, e.g. o-benzotriazol-1-yl-N,N,N′,N′-tetramethyluroniumhexafluorophosphate or O-(7-azabenzotriazol-1-yl)-N,N,N′,N′-tetramethyluronium hexafluorophosphate, in a solvent such as acetonitrile, dimethyl formamide, or a mixture thereof. A suitable base such as N,N-diisopropylethylamine may be used in the reaction, which can be performed at a temperature in the range of 0° C. to +20° C. Alternatively, a solution of VI in a solvent such as dimethyl acetamide can be first reacted with 1,1′-carbonylbis(1H-imidazole) at a temperature in the range of +80° C. to +120° C., and then reacted with the amine VII at a temperature in the range of +100° C. to +150° C., using an oil bath or a microwave oven.

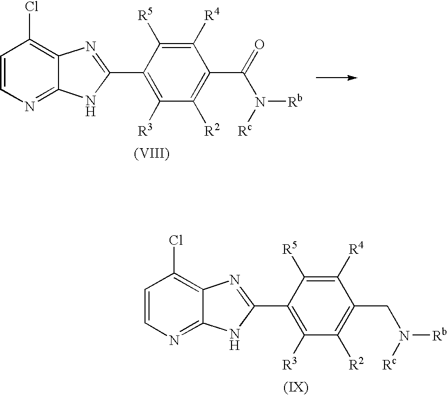

(v) A compound of type VIII can be transformed into a compound of type IX by reaction with a suitable reducing agent, e.g. borane, in a suitable solvent such as tetrahydrofuran, at a temperature in the range of 0° C. to +60° C.

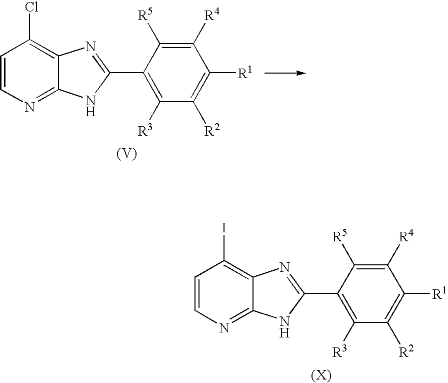

(vi) A compound of type V can be transformed into the corresponding iodide X by (a) first, treatment with HCl in a suitable solvent such as diethyl ether to give the hydrochloride salt, and (b) second, reaction of the salt with NaI in a suitable solvent, e.g. acetonitrile, at a temperature in the range of +150° C. to +175° C. using an oil bath or a microwave oven.

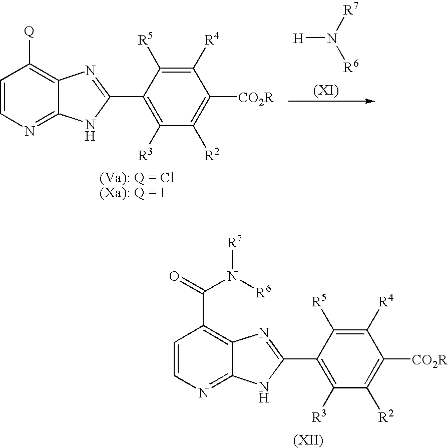

(vii) A compound of type Va or Xa may be converted into a carboxamide of type XII by reaction with an amine XI according to the following (wherein R is alkyl, for example methyl or ethyl).
(a) in the presence of a suitable catalyst, e.g. PdCl₂(dppf), suitable amine co-reagents such as 1,8-diazabicyclo[5.4.0]undec-7-ene and imidazole, and molybdenum hexacarbonyl, the reaction can be carried out in a suitable solvent such as THF by heating to a temperature in the range of +125° C. to +175° C. in a microwave oven;
(b) in the presence of a suitable catalyst, e.g. Pd(OAc)₂/1,3-bis(diphenylphosphino)propane or PdCl₂(BINAP), the reaction is run in an autoclave under a pressure of carbon monoxide of 1-5 bar, in a suitable solvent such as dioxane, and at a temperature in the range of +80° C. to +120° C.

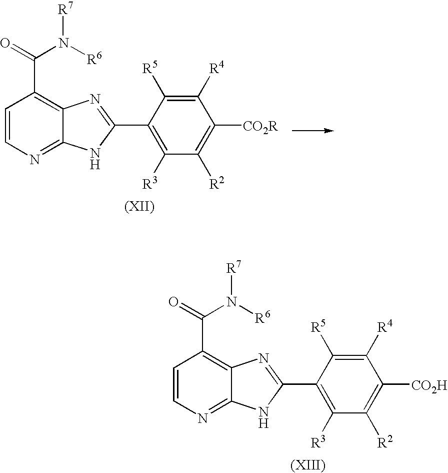

(viii) Hydrolysis of an ester of type XII to the corresponding acid XIII can be performed as described above for the conversion of Va to VI.

### Methods of Preparation of End Products

Another objective of the invention are processes for the preparation of a compound of general formula I, wherein R¹, R², R³, R⁴, R⁵, R⁶, R⁷, R^{b} and R^{c} are, unless specified otherwise, defined as in formula I, comprises of:

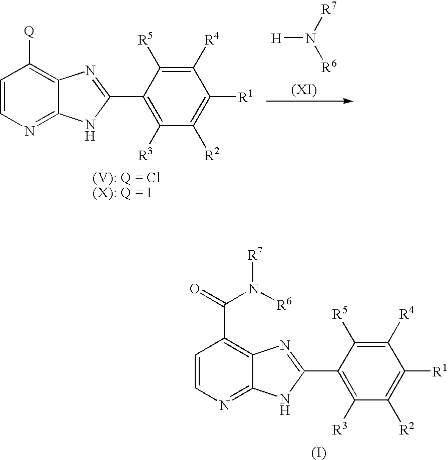

(i) A compound of type V or X may be coupled with an amine XI to give a compound of type I as describe above for the reaction of Va or Xa with XI to give XII.

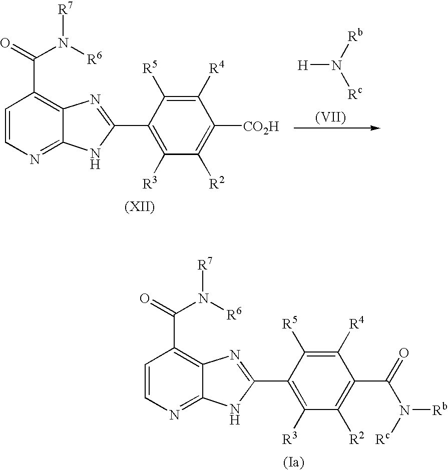

(ii) An ester of type XII may be transformed into a compound of type Ia (I, A=CONR^{b}R^{c}) by (a) first, heating neat with an amine VII at a temperature in the range of +180° C. to +220° C. using an oil bath or a microwave oven, and (b) second, after cooling, adding a suitable catalyst such as o-benzotriazol-1-yl-N,N,N′,N′-tetramethyluroniumhexafluorophosphate or O-(7-azabenzotriazol-1-yl)-N,N,N′,N′-tetramethyluronium hexafluorophosphate, and continuing the reaction at a temperature in the range of 0° C. to +20° C.

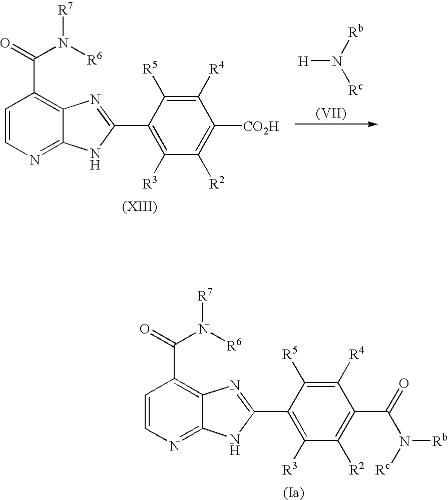

(iii) Coupling of a carboxylic acid of type XIII with an amine of type VII to give Ia can be performed as described above for the preparation of VIII from VI and VII.

Consequently, in one aspect of the present invention, there is provided a process for preparing a compound of formula I, wherein R¹, R², R³R⁴, R⁵, R⁶, R⁷, R^{b} and R^{c} are, unless specified otherwise, defined as in formula I, comprising of:

(i) Metal-catalyzed carbonylative coupling of a compound of type V or X with an amine XI, using molybdenum hexacarbonyl or carbon monoxide gas, optionally with added amine co-reagents.
(ii) An ester of type XII may be coupled with an amine VII to give a compound of type Ia (I, A=CONR^{b}R^{c}) by first heating XII with the neat amine VII, and then adding a suitable catalyst and continuing the reaction.
(iii) Formation of an amide of type Ia can also be performed by reacting a carboxylic acid of type XIII with an amine of type VII, in the presence of a suitable catalyst, optionally with an added amine base. Alternatively, the acid XIII can be first reacted with an activating agent, and then reacted with the amine.

The hydrochloric salt of a compound of formula I may be obtained from a compound of formula I by treatment with hydrochloric acid at a temperature range between 0° C. and +25° C., in a suitable solvent such as dichloromethane, tetrahydrofuran or dichloromethane/methanol mixture.

### General Methods

All solvents used were analytical grade and commercially available anhydrous solvents were routinely used for reactions. Reactions were typically run under an inert atmosphere of nitrogen or argon.

¹H, ¹⁹F and ¹³C NMR spectra were recorded at 400 MHz for proton, 376 MHz for fluorine-19 and 100 MHz for carbon-13, either on a Varian Unity+ 400 NMR Spectrometer equipped with a 5 mm BBO probehead with Z-gradients, or a Bruker Avance 400 NMR spectrometer equipped with a 60 μl dual inverse flow probehead with Z-gradients, or a Bruker DPX400 NMR spectrometer equipped with a 4-nucleus probehead equipped with Z-gradients, or a Bruker Avance 600 NMR spectrometer equipped with a 5 mm BBI probehead with Z-gradients. Unless specifically noted in the examples, spectra were recorded at 400 MHz for proton, 376 MHz for fluorine-19 and 100 MHz for carbon-13. The following reference signals were used: the middle line of DMSO-d₆ δ 2.50 (¹H), δ 39.51 (¹³C); the middle line of CD₃OD δ3.31 (¹H) or δ 49.15 (¹³C), CDCl₃ δ 7.26 (¹H) and the middle line of CDCl₃ δ 77.16 (¹³C) (unless otherwise indicated).

Mass spectra were recorded on a Waters LCMS consisting of an Alliance 2795 (LC), Waters PDA 2996 and a ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 100-700 with a scan time of 0.3 s. Separations were performed on either Waters X-Terra MS C8 (3.5 μm, 50 or 100 mm×2.1 mm i.d.) or an ACE 3 AQ (100 mm×2.1 mm i.d.) obtained from ScantecLab. Flow rates were regulated to 1.0 or 0.3 mL/min, respectively. The column temperature was set to 40° C. A linear gradient was applied using a neutral or acidic mobile phase system, starting at 100% A (A: 95:5 0.1M NH₄OAc:MeCN or 95:5 8 mM HCOOH:MeCN) ending at 100% B (MeCN).

Alternatively, mass spectra were recorded on a Waters LC-MS system (Sample Manager 2777C, 1525μ binary pump, 1500 Column Oven, ZQ, PDA2996 and ELS detector, Sedex 85). Separation was performed using a Zorbax column (C8, 3.0×50 mm, 3 μm). A four minutes linear gradient was used starting at 100% A (A: 95:5 10 mM NH₄OAc:MeOH) and ending at 100% B (MeOH). The ZQ was equipped with a combined APPI/APCI ion source and scanned in the positive mode between m/z 120-800 with a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode.

Alternatively, mass spectra were recorded on a Waters LCMS consisting of an Alliance 2690 Separations Module, Waters 2487 Dual 1 Absorbance Detector (220 and 254 nm) and a Waters ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 97-800 with a scan time of 0.3 or 0.8 s. Separations were performed on a Chromolith Performance RP-18e (100×4.6 mm). A linear gradient was applied starting at 95% A (A: 0.1% HCOOH (aq.)) ending at 100% B (MeCN) in 5 minutes. Flow rate: 2.0 mL/min.

Microwave heating was performed in a Creator or Smith Synthesizer Single-mode microwave cavity producing continuous irradiation at 2450 MHz.

HPLC analyses were performed on an Agilent HP 1000 system consisting of G1379A Micro Vacuum Degasser, G1312A Binary Pump, G1367A Well plate auto-sampler, G1316A Thermostatted Column Compartment and G1315B Diode Array Detector. Column: X-Terra MS, Waters, 3.0×100 mm, 3.5 μm. The column temperature was set to 40° C. and the flow rate to 1.0 ml/min. The Diode Array Detector was scanned from 210-300 nm, step and peak width were set to 2 nm and 0.05 min, respectively. A linear gradient was applied, starting at 100% A (95:5 10 mM NH₄OAc:MeCN) and ending at 100% B (B: acetonitrile), in 4 min.

A typical workup procedure after a reaction consisted of extraction of the product with a solvent such as ethyl acetate, washing with water followed by drying of the organic phase over MgSO₄ or Na₂SO₄, filtration and concentration of the solution in vacuo.

Thin layer chromatography (TLC) was performed on Merck TLC-plates (Silica gel 60 F₂₅₄) and UV visualized the spots. Flash chromatography was preformed on a Combi Flash® Companion™ using RediSep™ normal-phase flash columns. Typical solvents used for flash chromatography were mixtures of chloroform/methanol, dichloromethane/methanol, heptane/ethyl acetate, chloroform/methanol/ammonia (aq.) and dichloromethane/methanol/ammonia (aq.). SCX ion exchange columns were performed on Isolute® columns. Chromatography through ion exchange columns were typically performed in solvents such a methanol, or 10% ammonia in methanol. Preparative chromatography was run on a Waters autopurification HPLC with a diode array detector. Column: XTerra MS C8, 19×300 mm, 10 μm. Narrow gradients with MeCN/(95:5 0.1M NH₄OAc:MeCN) were used at a flow rate of 20 m/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Narrow gradients with MeCN/0.1% trifluoroacetic acid in MilliQ Water were used at a flow rate of 10 ml/min.

The formation of hydrochloride salts of the final products were typically performed by dissolution in solvents or solvent mixtures such as diethyl ether, tetrahydrofuran, dichloromethane/methanol, followed by addition of 1M HCl in diethyl ether.

The following abbreviations have been used:
AIBN 2,2′-azobis(2-methylpropionitrile);
aq. aqueous;
CH₂Cl₂ dimethyl chloride;
BINAP 2,2′-bis(diphenylphosphino)-1,1′binaphtyl;
DBU 1,8-Diazabicyclo[5.4.0]undec-7-ene;
DMF N—N-dimethylformamide;
ether diethyl ether;
Et₂O diethyl ether;
EtOAc ethyl acetate;
EtOH ethanol;
HBTU o-benzotriazol-1-yl-N,N,N′,N′-tetramethyluroniumhexafluorophosphate;
HCl hydrochloride;
HOAc acetic acid;
(i-Pr)₂NEt N—N-diisopropylethylamine;
m-CPBA 3-chloroperoxybenzoic acid;
MeCN acetonitrile;
MeOH methanol;
MgSO₄ magnesium sulphate;
Mo(CO)₆ molybdenum hexacarbonyl
NaHCO₃ sodium bicarbonate;
NaI sodium iodide;
Na₂SO₄ sodium sulphate;
Na₂S₂O₃ sodium thiosulphate
NH₄OAc ammonium acetate;
Pd(OAc)₂ palladium diacetate;
PdCl₂(dppf)*DCM (1,1′-bis(diphenylphosphino)ferrocen)palladiuln(II) chloride dichlorometane adduct;
Pd(dppf)Cl₂ 1,1′-bis(diphenylphosphino)ferrocene palladium (II) chloride;
PdCl₂(BINAP) 2,2′-bis(diphenylphosphine)-1,1′-binaphtyl palladium (II) dichloride
POCl₃ phosphoroxidchloride
r.t. room temperature;
THF tetrahydrofuran.

Starting materials used were either available from commercial sources or prepared according to literature procedures and had experimental data in accordance with those reported. The following is an example of a starting material that was prepared: 2-(Benzyloxy)-4-chloro-3-nitropyridine: Arvanitis, A. G., et al, Bioorganic & Medicinal Chemistry Letters, 2003, 13, 125-128.

Compounds have been named either using ACD/Name, version 8.08, software from Advanced Chemistry Development, Inc. (ACD/Labs), Toronto ON, Canada, www.acdlabs.com, 2004 or using Openeye lexichem version 1.4 (Copyright© 1997-2006 OpenEye Scientific Software, Santa Fe, N. Mex.) to generate the IUPAC name.

In the following general methods A to F, the groups R¹, R² and R³ are used independently to indicate the diversity of substitution within each structure. The identity of R¹, R² and R³ will be clear to a person skilled in the art based on the starting materials and intermediates for each specific example. For instance in Example 1, which refers to General method B, B1 is 7-chloro-2-[2-(trifluoromethyl)phenyl]-3H-imidazo[4,5-b]pyridine such that R¹ is 2-trifluoromethyl-, B2 is 7-iodo-2-[2-(trifluoromethyl)phenyl]-3H-imidazo[4,5-b]pyridine and B3 is 3-methoxypropylamine such that R² is hydrogen and R³ is 3-methoxypropyl-.

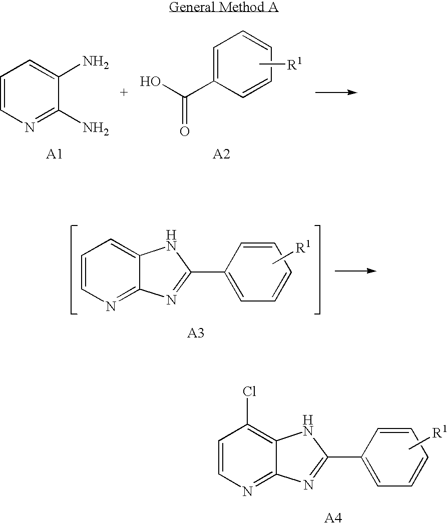

The acid A2 (1.0 equiv.) and HBTU (1.0 equiv.) were dissolved in a mixture of MeCN/DMF (8:2) under argon atmosphere. (i-Pr)₂NEt (3.0 equiv.) was added dropwise, and then 2,3-diaminopyridine A1 (1.0 equiv.) was added. The reaction mixture was stirred at r.t. overnight. The mixture was then poured onto a saturated solution of NaHCO₃ and the product was extracted with EtOAc. The organic layer was washed with saturated NaHCO₃ (aq.), water and saturated ammonium chloride (aq.). The organic layer was dried (Na₂SO₄), filtered and evaporated in vacuo to afford a crude product, which was diluted in HOAc. The solution was stirred at +180° C. for 10 minutes in a microwave reactor. The reaction mixture was cooled to r.t. and used in the next step without any further purification. m-CPBA (4.0 equiv.) was added to the acetic acid solution of the imidazopyridine A3 and the resulting mixture was stirred overnight at r.t. The solution was evaporated in vacuo and Et₂O was added. The mixture was filtered and the N-oxide, which precipitated, was washed with Et₂O. The solid was then dried in vacuo overnight at +40° C. A suspension of the solid in POCl₃ was heated in a microwave reactor for 10 minutes at +120° C. The solution was concentrated in vacuo and basified with saturated NaHCO₃ (aq.). The aqueous layer was extracted with EtOAc, and the organic layer was dried (Na₂SO₄), filtered and evaporated in vacuo to afford a crude solid which was used in the next step without further purification.

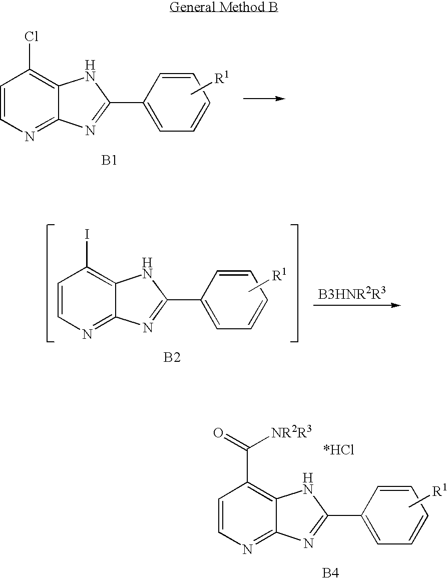

The imidazopyridine B1 (1.0 equiv.) was suspended in THF and 1M HCl in ether was added slowly. The solvents were removed in vacuo and the resulting salt was dried at +60° C. in vacuo. The salt was mixed with sodium iodide (10 equiv.) and acetonitrile was added. The reaction mixture was stirred at +160° C. for 10 minutes in a microwave reactor. After cooling to r.t., the mixture was poured onto a solution of Na₂S₂O₃ (10%) and saturated NaHCO₃ (aq.). The product was extracted with EtOAc. The organic layer was dried (Na₂SO₄), filtered and concentrated in vacuo to afford the iodoimidazopyridine B2 as a crude product.

The iodoimidazopyridine B2 (1.0 equiv.) was mixed with an amine B3 (4.0 equiv.), DBU (3.0 equiv.), imidazole (0.5 equiv.), Mo(CO)₆ (1.0 equiv) and PdCl₂(dppf)*DCM (0.1 equiv.) in THF. The reaction mixture was heated at +150° C. for 15 minutes in a microwave reactor. After cooling to r.t. the solvent was evaporated in vacuo. The crude product was diluted in MeOH and pre-purified with a SCX column (ion exchange resin), followed by purification by preparative HPLC. After extraction with EtOAc, drying (Na₂SO₄) and filtration, the solvent was evaporated in vacuo to afford a solid. This solid was dissolved in THF and 1M HCl in ether was added. The product hydrochloride salt was afforded after evaporation of the solvent in vacuo.

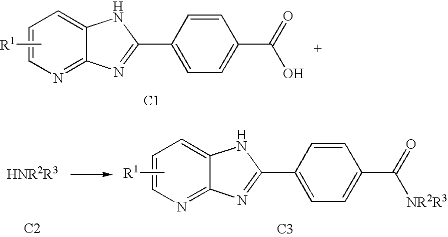

(i-Pr)₂NEt (3.0 equiv.) was added to a suspension of the benzoic acid C1 (1.0 equiv.), the amine C2 (1.2 equiv.) and HBTU (1.2 equiv.) in MeCN (5 mL) and the reaction mixture was stirred at r.t. for 30 minutes. Saturated NaHCO₃ (aq.) was added and the precipitated product was collected by filtration, washed with water and dried. The product was used in the next step without further purification.

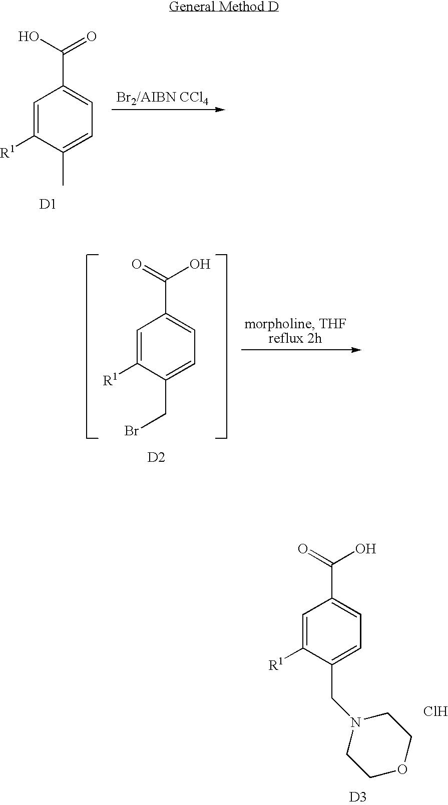

A solution of the acid D1 (1 eq) in CCl₄ (30 mL) was stirred at +80° C. AIBN (50 mg) was added. Bromine (1 eq) was added dropwise in 5 h. After cooling down to r.t. the solvent was removed under vacuum to afford the crude bromo substance D2. Morpholine (2.0 mL) was added to a suspension of the crude substance D2 in THF (100 mL) and the mixture was stirred at reflux for 2 h. The solution was cooled down to r.t. and the solvent evaporated under vacuum. The resulting solid was dissolved in NaOH 1N aqueous solution (50 mL) and this solution was extracted with CHCl₃ three times. The aqueous layer was acidified to pH 1 with concentrated aqueous HCl. This solution was concentrated under vacuum to 15 mL and then filtered. The obtained salt D3 was dried under vacuum for 15 h.

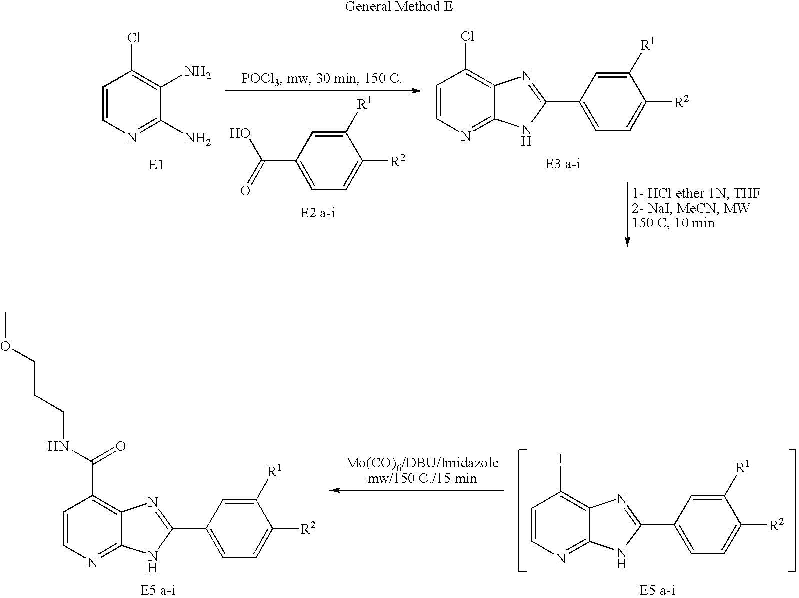

2,3-Diamino-4-chloropyridine E1 (prepared according to EP0420237) (143 mg, 1.0 mmol) and the amino acid E2a-i (1.0-1.1 mmol) were dissolved in POCl₃ (15 mL) in a 20 mL microwave vial. The reaction mixture was stirred at +150° C. for 30 min in a microwave reactor. After cooling down, the solvent was removed under vacuum. A saturated solution of NaHCO₃ was added and this solution was extracted with EtOAc. The organic layer was dried over Na₂SO₄, filtered and evaporated under vacuum to afford E3a-i as a crude product.

### General Procedure for Amidation:

The imidazopyridine E3a-i (1.0 equiv.) was suspended in THF and 1N HCl in ether was added slowly. The solvents were removed in vacuo and the resulting salt was dried at +60° C. in vacuo. The salt was mixed with NaI (10 equiv.) and MeCN was added. The reaction mixture was stirred at +160° C. for 10 minutes in a microwave reactor. After cooling to r.t., the mixture was poured onto a solution of Na₂S₂O₃ (10%) and saturated NaHCO₃ (aq.). The product was extracted with EtOAc. The organic layer was dried (Na₂SO₄), filtered and concentrated in vacuo to afford the iodoimidazopyridine E4a-i as a crude product.

This iodoimidazopyridine E4a-i (1.0 equiv.) was mixed with 3-methoxypropylamine (4.0 equiv.), DBU (3.0 equiv.), imidazole (0.5 equiv.) Mo(CO)₆ (1.0 equiv) and Pd(dppf)Cl₂ (0.1 equiv.) in THF. The reaction mixture was heated at +150° C. for 15 minutes in a microwave reactor. After cooling to r.t. the solvent was evaporated in vacuo. The crude product was diluted in MeOH and pre-purified with a SCX column (ion exchange resin), followed by purification by preparative HPLC. After extraction with EtOAc, drying (Na₂SO₄) and filtration the solvent was evaporated in vacuo to afford a solid. This solid was dissolved in THF and 1N HCl in ether was added. The title compound E5a-i was afforded after evaporation of the solvent in vacuo.

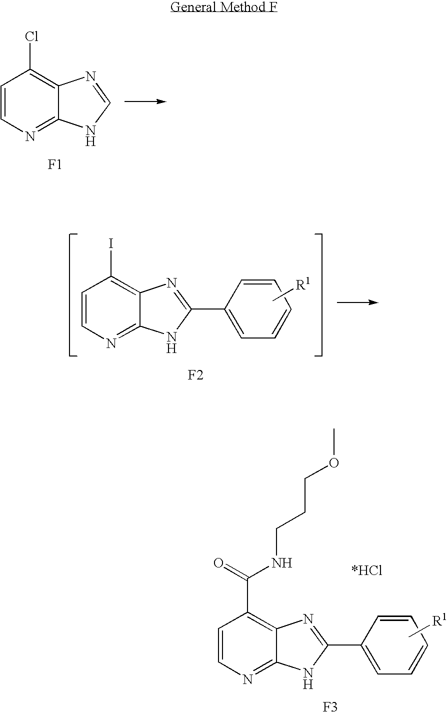

The imidazopyridine F1 (1.0 equiv.) was suspended in THF and 1N HCl in ether was added slowly. The solvents were removed in vacuo and the resulting salt was dried at +60° C. in vacuo. The salt was mixed with NaI (10 equiv.) and CH₃CN was added. The reaction mixture was stirred at +160° C. for 10 minutes in a microwave reactor. After cooling to r.t., the mixture was poured onto a solution of Na₂S₂O₃ (10%) and saturated NaHCO₃ (aq.).

The product was extracted with EtOAc. The organic layer was dried (Na₂SO₄), filtered and concentrated in vacuo to afford the iodoimidazopyridine F2 as a crude product. The iodoimidazopyridine F2 (1.0 equiv.) was mixed with 3-methoxypropylamine (4.0 equiv.), DBU (3.0 equiv.), imidazole (0.5 equiv.) Mo(CO)₆ (1.0 equiv) and Pd(dppf)Cl₂ (0.1 equiv.) in THF. The reaction mixture was heated at +150° C. for 15 minutes in a microwave reactor. After cooling to r.t. the solvent was evaporated in vacuo. The crude product was diluted in MeOH and pre-purified with a SCX column (ion exchange resin), followed by purification by preparative HPLC. After extraction with EtOAc, drying (Na₂SO₄) and filtration the solvent was evaporated in vacuo to afford a solid. This solid was dissolved in THF and 1N HCl in ether was added. The title compound was afforded after evaporation of the solvent in vacuo.

### WORKING EXAMPLES

Below follows a number of non-limiting examples of the compounds of the present invention.

### Example 1

### N-(3-Methoxypropyl)-2-[2-(trifluoromethyl)phenyl]-3H-imidazo[4,5-b]pyridine-7-carboxamide hydrochloride

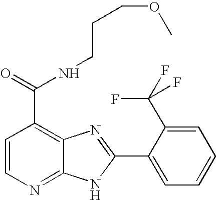

The title compound was prepared in accordance with the general method B using 7-chloro-2-[2-(trifluoromethyl)phenyl]-3H-imidazo[4,5-b]pyridine (45 mg, 0.15 mmol, obtained from Example 1(a)) and 3-methoxypropylamine (55 mg, 0.62 mmol), affording 19 mg (30%) of the title compound.

¹H NMR (CD₃OD) δ ppm 8.82 (d, J=5.8 Hz, 1H), 8.09-8.00 (m, 2H), 7.98-7.87 (m, 3 H), 3.63-3.56 (m, 2H), 3.53 (t, J=6.1 Hz, 2H), 3.35 (s, 3H), 1.98-1.92 (m, 2H); ¹⁹F NMR (376 MHz, CD₃OD) δ ppm −60.21 (s, 3 F); MS (ESI) m/z 379 (M+1).

### Example 1 (a)

### 7-Chloro-2-[2-(trifluoromethyl)phenyl]-3H-imidazo[4,5-b]pyridine

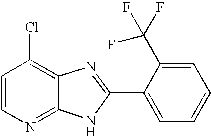

The title compound was prepared in accordance with the general method A using 2,3-diaminopyridine (0.272 g, 2.5 mmol) and 2-(trifluoromethyl)benzoic acid (0.475 g, 2.5 mmol). After purification by preparative HPLC, the title compound was afforded in 0.545 g (73%) yield.

MS (ESI) m/z 298 (M+1).

### Example 2

### N-(3-Methoxypropyl)-2-[3-(trifluoromethyl)phenyl]-3H-imidazo[4,5-b]pyridine-7-carboxamide hydrochloride

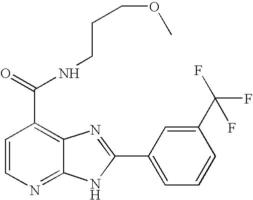

The title compound was prepared in accordance with the general method B using 7-chloro-2-[3-(trifluoromethyl)phenyl]-3H-imidazo[4,5-b]pyridine (80 mg, 0.27 mmol, obtained from Example 2(a)) and 3-methoxypropylamine (65 mg, 0.73 mmol), affording 8 mg (7%) of the title compound.

¹H NMR (CD₃OD) δ ppm 8.68 (s, 1H), 8.66 (d, J=5.6 Hz, 1H), 8.59 (d, J=7.8 Hz, 1 H), 7.99 (d, J=7.8 Hz, 1H), 7.93 (d, J=5.6 Hz, 1H), 7.87 (t, J=7.8 Hz, 1H), 3.65 (t, J=6.8 Hz, 2H), 3.58 (t, J=6.1 Hz, 2H), 3.37 (s, 3H), 2.02-1.96 (m, 2H); ¹⁹F NMR (376 MHz, CD₃ OD) δ ppm −64.82 (s, 3 F); MS (ESI) m/z 379 (M+1).

### Example 2(a)

### 7-Chloro-2-[3-(trifluoromethyl)phenyl]-3H-imidazo[4,5-b]pyridine

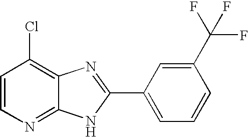

The title compound was prepared in accordance with the general method A using 2,3-diaminopyridine (0.272 g, 2.5 mmol) and 3-(trifluoromethyl)benzoic acid (0.475 g, 2.5 mmol), affording a crude yield of 0.545 g (73%).

MS (ESI) m/z 298 (M+1).

### Example 3

### N-(3-Methoxypropyl)-2-[4-(trifluoromethyl)phenyl]-3H-imidazo[4,5-b]pyridine-7-carboxamide hydrochloride

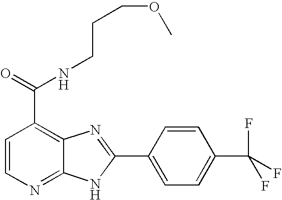

The title compound was prepared in accordance with the general method B using 7-chloro-2-[4-(trifluoromethyl)phenyl]-3H-imidazo[4,5-b]pyridine (80 mg, 0.27 mmol, obtained from Example 3(a)) and 3-methoxypropylamine (61 mg, 0.68 mmol), affording 14 mg (20%) of the title compound.

¹H NMR (CD₃OD) δ ppm 8.70 (d, J=5.8 Hz, 1H), 8.51 (d, J=8.3 Hz, 2H), 7.97 (d, J=8.3 Hz, 2H), 7.95 (d, J=8.3 Hz, 1H), 3.64 (t, J=6.9 Hz, 2H), 3.57 (t, J=6.1 Hz, 2H), 3.37 (s, 3H), 2.12-1.82 (m, 2H); ¹⁹F NMR (376 MHz, CD₃OD) δ ppm −65.09 (s, 3 F);

MS (ESI) m/z 379 (M+1).

### Example 3(a)

### 7-Chloro-2-[4-(trifluoromethyl)phenyl]-3H-imidazo[4,5-b]pyridine

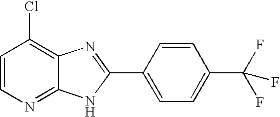

The title compound was prepared in accordance with the general method A using 2,3-diaminopyridine (0.272 g, 2.5 mmol) and 4-(trifluoromethyl)benzoic acid (0.475 g, 2.5 is mmol), affording a crude yield of 0.514 g (69%).

MS (ESI) m/z 298 (M+1).

### Example 4

### N-(3-Methoxypropyl)-2-{3-[(2,2,3,3-tetrafluoropropoxy)methyl]phenyl}-3H-imidazo[4,5-b]pyridine-7-carboxamide hydrochloride

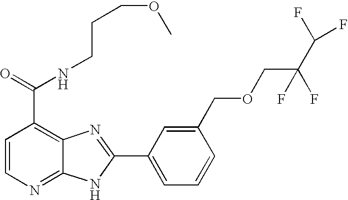

The title compound was prepared in accordance with the general method B using 7-chloro-2-{3-[(2,2,3,3-tetrafluoropropoxy)methyl]phenyl}-3H-imidazo[4,5-b]pyridine (0.103 g, 0.27 mmol, obtained from Example 4(a)) and 3-methoxypropylamine (82 mg, 0.912 mmol), affording 30 mg (20%) of the title compound.

¹HNMR (CD₃OD) δ ppm 8.71 (d, J=5.8 Hz, 1H), 8.32 (s, 1H), 8.26 (d, J=7.6 Hz, 1 H), 7.93 (d, J=5.8 Hz, 1H), 7.78-7.67 (m, 2H), 6.45-5.98 (m, 1H), 4.82 (s, 2H), 4.10-3.89 (m, 2H), 3.68-3.59 (m, 2H), 3.58-3.51 (m, 2H), 3.37 (s, 3H), 2.05-1.87 (m, 2H); ¹⁹F NMR (376 MHz, CD₃OD) δ ppm −127.23-−128.61 (m, 2 F), −142.10 (d, J=52.8 Hz, 2 F); MS (ESI) m/z 374 (M+1).

### Example 4(a)

### 7-Chloro-2-{3-[(2,2,3,3-tetrafluoropropoxy)methyl]phenyl}-3H-imidazo[4,5-b]pyridine

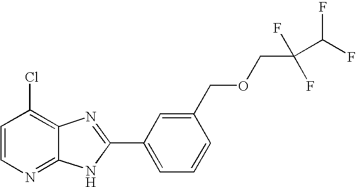

The title compound was prepared in accordance with the general method A using 2,3-diaminopyridine (0.272 g, 2.5 mmol) and 3-[2,2,3,3-tetrafluoropropoxy)methyl]benzoic acid (0.665 g, 2.5 mmol). After purification with preparative HPLC, the title compound was afforded in 0.217 g (23%) yield.

MS (ESI) m/z 374 (M+1).

### Example 5

### N-(3-Methoxypropyl)-2-[4-(morpholin-4-ylmethyl)phenyl]-3H-imidazo[4,5-b]pyridine-7-carboxamide hydrochloride

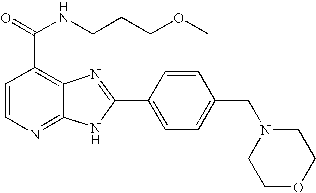

The product mixture from the preparation of 7-iodo-2-[4-(morpholin-4-ylmethyl)phenyl]-3H-imidazo[4,5-b]pyridine (0.366 mmol, obtained from Example 5(f)) was mixed with 3-methoxypropan-1-amine (5 mL), 1,3-bis(diphenylphosphino)propane (9 mg, 0.022 mmol), Pd(OAc)₂ (4 mg, 0.018 mmol) in 1,4-dioxane in an autoclave and purged with nitrogen followed by carbon monoxide (g). The vessel was pressurized to 5 bar with carbon monoxide (g) and heated to +100° C. for 2 h. The reaction mixture was allowed to cool to r.t., filtered through diatomaceous earth, and the solvent was evaporated in vacuo. The residue was purified by preparative HPLC, which afforded the product as a base. The hydrochloride salt was prepared by dissolving the base in CH₂Cl₂/MeOH (9:1) and 1M HCl in Et₂O was added until precipitation formed. The hydrochloride salt was collected by filtration and dried, affording 69 mg (37%) of the title compound.

¹H NMR (DMSO-d₆) δ ppm 11.85 (m, 1H), 8.51 (d, J=5.1 Hz, 1H), 8.42 (d, J=8.6 Hz, 2H), 7.90 (d, J=8.3 Hz, 2H), 7.75 (d, J=5.1 Hz, 1H), 4.43 (d, J=4.5 Hz, 2H), 4.01-3.75 (m, 4H), 3.61-3.45 (m, 4H), 3.28 (s, 3H), 3.27-3.23 (m, 2H), 3.16 (s, 1H), 3-15-3.09 (m, 2H), 1-97-1.78 (m, 2H); MS (APPI) m/z 410 (M+1).

### Example 5(a)

### Methyl 4-(3H-imidazo[4,5-b]pyridin-2-yl)benzoate

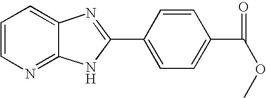

(i-Pr)₂NEt (24 mL, 138 mmol) was added to a suspension of pyridine-2,3-diamine (5.0 g, 45.9 mmol), terephtalic acid monomethyl ester (8.26 g, 45.9 mmol) and HBTU (20.9 g, 55.0 mmol) in MeCN (200 mL) and the reaction mixture was stirred at r.t. for 1 h. A precipitate that formed was collected and washed with MeCN. The solid was distributed into microwave vials with HOAc (4 mL) and heated to +200° C. for 5 minutes. The product precipitated at r.t., filtered, washed with HOAc and MeCN and dried to afford 9.6 g (83% yield) of the title compound.

MS (ESI) m/z 254 (M+1).

### Example 5(b)

### Methyl 4-(7-chloro-3H-imidazo[4,5-b]pyridin-2-yl)benzoate

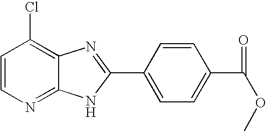

Methyl 4-(3H-imidazo[4,5-b]pyridin-2-yl)benzoate (8.3 g, 32.8 mmol, obtained from Example 5(a)) and m-CPBA (70%, 22 g, 98.4 mmol) in HOAc was stirred at r.t. for 18 h. The solvent was evaporated in vacuo and the residue was crystallized from EtOH. The solid was mixed with POCl₃ and heated in a microwave reactor at +120° C. for 5 minutes. After cooling to r.t., the mixture was poured into ice/water mixture and the precipitate that formed was collected, washed with water and dried, affording the title compound in 8.0 g (85%) yield.

¹H NMR (DMSO-d₆) δ ppm 8.46-8.39 (m, 2H), 8.34 (d, J=5.3 Hz, 1H), 8.17-8.10 (m, 2H), 7.46 (d, J=5.3 Hz, 1H), 3.90 (d, 3H); MS (ESI) m/z 288 (M+1).

### Example 5(c)

### 4-(7-Chloro-3H-imidazo[4,5-b]pyridin-2-yl)benzoic acid

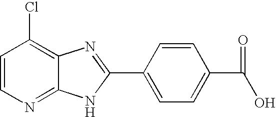

A mixture of methyl 4-(7-chloro-3H-imidazo[4,5-b]pyridin-2-yl)benzoate (7.7 g, 26.8 mmol, obtained from Example 5(b)) and lithium hydroxide (6.0 g, 250 mmol) in THF/water (9:1) was heated in microwave reactor at +120° C. for 10 minutes. After cooling to r.t. the mixture was made neutral using 2M HCl (aq.). The precipitate was filtered, washed with water and dried to afford the title compound in 7.0 g (96%) yield.

¹H NMR (DMSO-d₆) δ ppm 8.28 (d, J=8.3 Hz, 2H), 8.23 (d, J=5.3 Hz, 1H), 8.07 (d, J=8.1 Hz, 2H), 7.34 (d, J=5.3 Hz, 1H); MS (APPI) m/z 274 (M+1).

### Example 5(d)

### 7-Chloro-2-[4-(morpholin-4-ylcarbonyl)phenyl]-3H-imidazo[4,5-b]pyridine

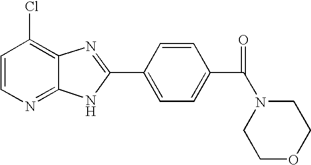

The title compound was prepared in accordance with the general method C using 4-(7-chloro-3H-imidazo[4,5-b]pyridin-2-yl)benzoic acid (1.0 g, 3.66 mmol, obtained from Example 5(c)) and morpholine (0.38 g, 4.39 mmol), affording a crude yield of 1.67 g. The product was used without further purification in the next step.

¹H NMR (DMSO-d₆) δ ppm 8.33 (d, J=8.1 Hz, 2H), 8.30 (d, J=5.1 Hz, 1H), 7.62 (d, J=8.3 Hz, 2H), 7.42 (d, J=5.3 Hz, 1H), 3.80-3.20 (m, 8H);

MS (APPI) m/z 343 (M+1).

### Example 5(e)

### 7-Chloro-2-[4-(morpholin-4-ylmethyl)phenyl]-3H-imidazo[4,5-b]pyridine

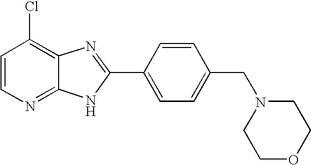

Borane-THF complex (1M, 20 mL) was added to 7-chloro-2-[4-(morpholin-4-ylcarbonyl)phenyl]-3H-imidazo[4,5-b]pyridine (1.7 g, 4.9 mmol, obtained from Example 5(d)) at r.t. After stirring at r.t. for 45 minutes, MeOH (200 mL) was added dropwise to the reaction mixture and the mixture was stirred for 2 h at r.t. The solvent was evaporated in vacuo, affording a crude of the title compound in 1.0 g (67%) yield. The crude product was used in the next step without further purification.

MS (APPI) m/z 329 (M+1).

### Example 5(f)

### 7-Iodo-2-[4-(morpholin-4-ylmethyl)phenyl]-3H-imidazo[4,5-b]pyridine

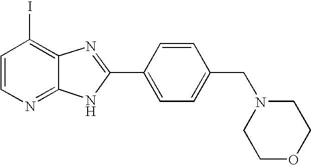

7-Chloro-2-[4-(morpholin-4-ylmethyl)phenyl]-3H-imidazo[4,5-b]pyridine (0.120 g, 0.366 mmol, obtained from Example 5(e)) was dissolved in CH₂Cl₂/MeOH (9:1, 5 mL), and HCl (1M in Et₂O, 2 mL) was added followed by addition of Et₂O until precipitation formed. The solid was collected by filtration and dried. The hydrochloride was mixed with sodium iodide (0.549 g, 3.66 mmol) and MeCN 10 (mL) and heated in a microwave reactor at +160° C. for 10 minutes. The mixture was diluted with CH₂Cl₂ (100 mL) and washed with Na₂S₂O₃ (10%, aq.) and brine. The organic phase was dried (Na₂SO₄), filtered and evaporated in vacuo. The remaining residue was a mixture of the title compound and starting material. The mixture was used in the next step without further purification.

MS (APPI) m/z 421 (M+1).

### Example 6

### N-(3-Methoxypropyl)-2-{4-[(4-methylpiperazin-1-yl)carbonyl]phenyl}-3H-imidazo[4,5-b]pyridine-7-carboxamide hydrochloride

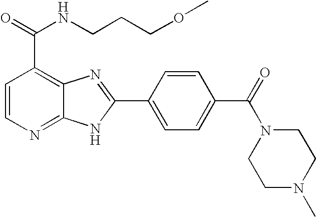

The base of the title compound was prepared in accordance with the general method C but with the exception that the reaction mixture was diluted with CH₂Cl₂ (50 mL) before extraction. Using 4-(7-{[(3-methoxypropyl)amino]carbonyl}-3H-imidazo[4,5-h]pyridin-2-yl)benzoic acid (50 mg, 0.141 mmol, obtained from Example 6(a)) and N-methylpiperazine (17 mg, 0.169 mmol) the base of the title compound was obtained, which was then purified by preparative HPLC. The hydrochloride salt was prepared by dissolving the base in CH₂Cl₂/MeOH (9:1) and 1M HCl in Et₂O was added until precipitation formed. The hydrochloride salt was collected by filtration and dried, affording 18 mg (25%) of the title compound.

¹H NMR (CD₃OD) δ ppm 8.66 (d, J=5.3 Hz, 1H), 8.44 (d, J=8.1 Hz, 2H), 7.92 (d, J=5.6 Hz, 1H), 7.78 (d, J=8.1 Hz, 2H), 3.64 (t, J=6.8 Hz, 2H), 3.80-3.39 (m, 8H), 3.37 (s, 3H), 3.20 (m, 2H), 2.98 (s, 3H), 2.10-1.88 (m, 2H).

MS (ESI) m/z 437 (M+1).

### Example 6(a)

### 4-(7-{[(3-Methoxypropyl)amino]carbonyl}-3H-imidazo[4,5-b]pyridin-2-yl)benzoic acid

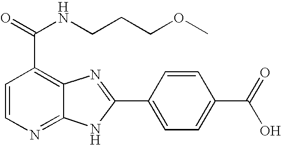

Methyl 4-(7-chloro-3H-imidazo[4,5-b]pyridin-2-yl)benzoate (obtained from Example 5(b)) (0.200 g, 0.697 mmol), R,S-(BINAP)PdCl₂ (0.084 g, 0.105 mmol), 3-methoxypropan-1-amine (10 mL) and 1,4-dioxane (50 mL) were mixed in an autoclave and purged with nitrogen followed by carbon monoxide (g). The vessel was pressurized to 5 bar with carbon monoxide (g) and heated to +100° C. for 48 h. The reaction mixture was allowed to cool to r.t., filtered through diatomaceous earth and the solvent was evaporated in vacuo. The crude product and lithium hydroxide (0.200 g, 8.3 mmol) were mixed in THF/water (9:1, 4 mL) and heated in a microwave reactor at +120° C. for minutes. After cooling to r.t. the reaction mixture was adjusted to neutral pH with 2M HCl. The precipitated solid was collected by filtration, to afford 0.157 g (63%) of the title compound.

MS (ESI) m/z 355 (M+1).

### Example 7

### N-(3-Methoxypropyl)-2-[4-(morpholin-4-ylcarbonyl)phenyl]-3H-imidazo[4,5-b]pyridine-7-carboxamide hydrochloride

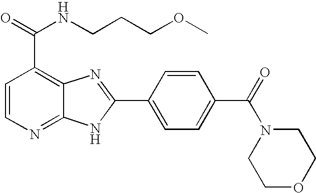

The base of the title compound was prepared in accordance with the general method C using 4-(7-{[(3-methoxypropyl)amino]carbonyl}-3H-imidazo[4,5-b]pyridin-2-yl)benzoic acid (obtained from Example 6(a)) (50 mg, 0.141 mmol) and morpholine (15 mg, 0.169 mmol). The base product was purified by preparative HPLC, and the hydrochloride salt was prepared by dissolving the base in CH₂Cl₂/MeOH (9:1) and 1M HCl in Et₂O was added until precipitation formed. The hydrochloride salt was collected by filtration and dried, affording 17 mg (24%) of the title compound.

¹H NMR (CD₃OD) δ ppm 8.57 (d, J=5.3 Hz, 1H), 8.37 (d, J=8.3 Hz, 2H), 7.91 (d, J=5.6 Hz, 1H), 7.65 (d, J=8.3 Hz, 2H), 3.65 (t, J=6.8 Hz, 2H), 3.89-3.60 (m, 6H), 3.57 (q, J=5.9 Hz, 2H), 3.49 (m, 2H), 3.37 (s, 3H), 2.08-1.91 (m, 2H);

MS (ESI) m/z 424 (M+1).

### Example 8

### 2-[3-Fluoro-4-(morpholin-4-ylmethyl)phenyl]-N-(3-methoxypropyl)-3H-imidazo[4,5-b]pyridine-7-carboxamide hydrochloride

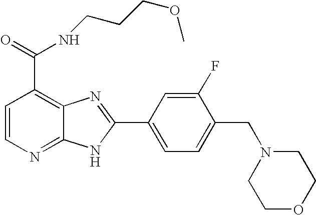

The title compound was prepared in accordance with the general method E using 7-chloro-2-[3-fluoro-4-(morpholin-4-ylmethyl)phenyl]-3H-imidazo[4,5-b]pyridine (obtained from Example 8(b)) (262 mg, 0.60 mmol), giving 27 mg (9% yield) of the title compound.

¹H NMR (400 MHz, DMSO-d₆) δ ppm 11.44 (s, 1H), 9.50 (s, 1H), 8.53 (d, 1H), 8.30 (t, 2H), 8.13-7.90 (m, 1H), 7.75 (d, 1H), 4.48 (s, 2H), 3.94 (s, 2H), 3.83 (s, 2H), 3.29 (s, 3H), 2.03-1.76 (m, 2H). ¹⁹F NMR (376 MHz, DMSO-d₆) δ ppm −113.48 (s, 1 F). MS (ESI) m/z 428 (M+1).

### Example 8(a)

### 3-Fluoro-4-(morpholin-4-ylmethyl)benzoic acid hydrochloride

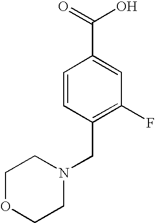

The title compound was prepared in accordance with the general method D using 3-fluoro-4-methylbenzoic acid (2.31 g, 15.0 mmol), giving 2.5 g (60% yield) of the title compound.

MS (APPI) m/z 240 (M+1).

### Example 8(b)

### 7-Chloro-2-[3-fluoro-4-(morpholin-4-ylmethyl)phenyl]-3H-imidazo[4,5-b]pyridine

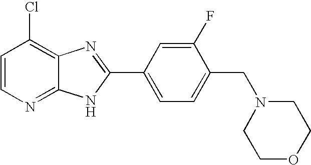

The title compound was prepared in accordance with the general method E using 3-fluoro-4-(morpholin-4-ylmethyl)benzoic acid hydrochloride (obtained from Example 8(a)) (302 mg, 1.1 mmol), giving 220 mg (63% yield) of the title compound.

MS (ESI) m/z 347 (M+1).

### Example 9

### 2-[3-Methoxy-4-(morpholin-4-ylmethyl)phenyl]-N-(3-methoxypropyl)-3H-imidazo[4,5-b]pyridine-7-carboxamide hydrochloride

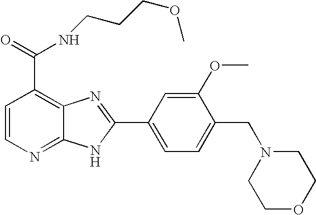

The title compound was prepared in accordance with the general method E using 7-chloro-2-[3-methoxy-4-(morpholin-4-ylmethyl)phenyl]-3H-imidazo[4,5-b]pyridine (obtained from Example 9(b)) (150 mg, 0.33 mmol), giving 20 mg (12% yield) of the title compound.

¹H NMR (400 MHz, MeOH) δ ppm 8.68 (d, 1H), 8.10 (s, 1H), 8.03 (d, 1H), 7.93 (d, 1 H), 7.79 (d, 1H), 7.75-7.54 (m, 1H), 4.51 (s, 2H), 4.12 (s, 3H), 4.06 (d, 2H), 3.85 (t, 2H), 3.64 (t, 2H), 3.57 (t, 2H), 3.47 (d, 2H), 3.36 (s, 3H), 1.99 (m, 2H). MS (ESI) m/z 440 (M+1).

### Example 9(a)

### 3-Methoxy-4-(morpholin-4-ylmethyl)benzoic acid hydrochloride

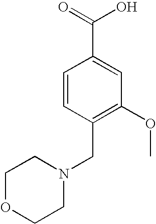

The title compound was prepared from 3-methoxy-4-bromomethylbenzoic methyl ester according to the procedure disclosed in WO9725033A1.

### Example 9(b)

### 7-Chloro-2-[3-methoxy-4-(morpholin-4-ylmethyl)phenyl]-3H-imidazo[4,5-b]pyridine

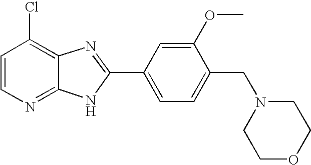

The title compound was prepared in accordance with the general method E using 3-methoxy-4-(morpholin-4-ylmethyl)benzoic acid hydrochloride (obtained from Example 9(a)) (288 mg, 1.0 mmol), giving 256 mg (71% yield) of the title compound.

MS (ESI) m/z 359 (M+1).

### Example 10

### N-(3-Methoxypropyl)-2-[4-(morpholin-4-ylmethyl)-3-(trifluoromethyl)phenyl]-3H-imidazo[4,5-b]pyridine-7-carboxamide hydrochloride

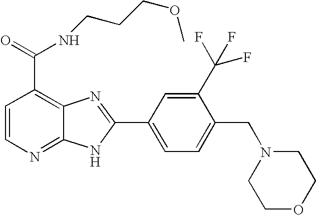

The title compound was prepared in accordance with the general method E using 7-chloro-2-[4-(morpholin-4-ylmethyl)-3-(trifluoromethyl)phenyl]-3H-imidazo[4,5-b]pyridine (obtained from Example 10(b)) (195 mg, 0.40 mmol), giving 22 mg (10% yield) of the title compound.

¹H NMR (400 MHz, MeOH) δ ppm 8.84 (s, 1H), 8.72 (d, 1H), 8.65 (d, 1H), 8.39 (d, 1 H), 7.93 (d, 1H), 4.72 (s, 2H), 4.17-3.83 (m, 4H), 3.65 (t, 2H), 3.59 (t, 2H), 3.51 (s, 4H), 3.37 (s, 3H), 2.10-1.85 (m, 2H).

MS (ESI) m/z 478 (M+1).

### Example 10(a)

### 4-(Morpholin-4-ylmethyl)-3-(trifluoromethyl)benzoic acid hydrochloride

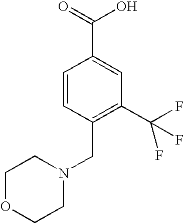

The title compound was prepared in accordance with the general method D using 4-methyl-3-trifluoromethylbenzoic acid (3.49 g, 14.54 mmol), giving 2.5 g (54% yield) of the title compound.

MS (ESI) m/z 290 (M+1).

### Example 10(b)

### 7-Chloro-2-[4-(morpholin-4-ylmethyl)-3-(trifluoromethyl)phenyl]-3H-imidazo[4,5-b]pyridine

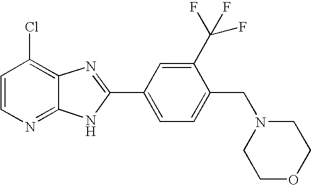

The title compound was prepared in accordance with the general method E using 3-trifluoromethyl-4-(morpholin-4-ylmethyl)benzoic acid hydrochloride (obtained from Example 10(a)) (358 mg, 1.1 mmol), giving 301 mg (76% yield) of the title compound.

MS (ESI) m/z 397 (M+1).

### Example 11

### N-(3-Methoxypropyl)-2-[4-(pyrrolidin-1-ylsulfonyl)phenyl]-3H-imidazo[4,5-b]pyridine-7-carboxamide hydrochloride

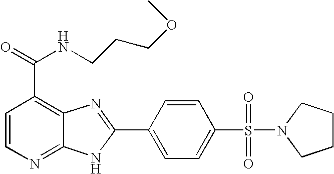

The title compound was prepared in accordance with the general method E using 7-chloro-2-[4-(pyrrolidin-1-ylsulfonyl)phenyl]-3H-imidazo[4,5-b]pyridine (obtained from Example 11(a)) (66 mg, 0.145 mmol), giving 6 mg (9% yield) of the title compound.

¹H NMR (400 MHz, DMSO-d₆) δ ppm 14.32 (s, 1H), 9.53 (s, 1H), 8.83-8.30 (m, 3H), 8.03 (d, 2H), 7.85-7.68 (m, 1H), 3.76-3.42 (m, 4H), 3.22 (t, 2H), 2.14-1.78 (m, 2 H), 1.74-1.57 (m, 4H), 1.26 (d, 2H). MS (ESI) m/z 444 (M+1).

### Example 11(a)

### 7-Chloro-2-[4-(pyrrolidin-1-ylsulfonyl)phenyl]-3H-imidazo[4,5-b]pyridine

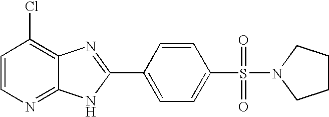

The title compound was prepared in accordance with the general method E using 4-(pyrrolidin-1-ylsulfonyl)benzoic acid (255 mg, 1.0 mmol), giving 65 mg (18% yield) of the title compound.

MS (ESI) m/z 363 (M+1).

### Example 12

### N-(3-Methoxypropyl)-2-{4-[(4-methylpiperazin-1-yl)sulfonyl]phenyl}-3H-imidazo[4,5-b]pyridine-7-carboxamide hydrochloride

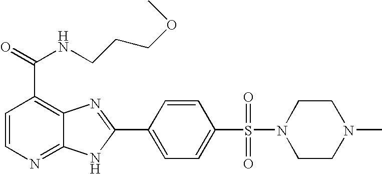

The title compound was prepared in accordance with the general method E using 7-chloro-2-{4-[(4-methylpiperazin-1-yl)sulfonyl]phenyl}-3H-imidazo[4,5-b]pyridine (obtained from Example 12(a)) (110 mg, 0.23 mmol), giving 13 mg (10% yield) of the title compound.

¹H NMR (400 MHz, DMSO-d₆) δ ppm 10.83 (s, 1H), 9.48 (t, 1H), 8.64 (d, 2H), 8.55 (d, 1H), 8.02 (d, 2H), 7.76 (d, 1H), 3.85 (d, 2H), 3.28 (s, 3H), 3.17 (d, 2H), 2.87-2.68 (m, 6H), 1.90 (m, 2H). MS (ESI) m/z 473 (M+1).

### Example 12(a)

### 7-Chloro-2-{4-[(4-methylpiperazin-1-yl)sulfonyl]phenyl}-3H-imidazo[4,5-b]pyridine

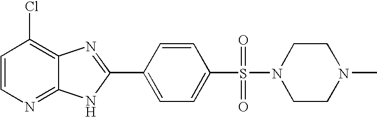

The title compound was prepared in accordance with the general method E using 4-[(4-methylpiperazin-1-yl)sulfonyl]benzoic acid (284 mg, 1.0 mmol), giving 195 mg (50% yield) of the title compound.

MS (ESI) m/z 392 (M+1).

### Example 13

### 2-{4-[(1,1-Dioxidothiomorpholin-4-yl)methyl]phenyl}-N-(3-methoxypropyl)-3H-imidazo[4,5-b]pyridine-7-carboxamide hydrochloride

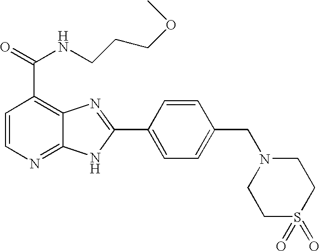

The title compound was prepared in accordance with the general method E using 7-chloro-2-{4-[(1,1-dioxidothiomorpholin-4-yl)methyl]phenyl}-3H-imidazo[4,5-b]pyridine (obtained from Example 13(a)) (120 mg, 0.25 mmol), giving 3 mg (2% yield) of the title compound.

¹H NMR (400 MHz, DMSO-d₆) δ ppm 9.58 (s, 1H), 8.46 (d, 1H), 8.32 (d, 2H), 7.76-7.64 (m, 2H), 7.61 (d, 2H), 3.88 (s, 2H), 3.62-3.44 (m, 2H), 3.28 (s, 3H), 3.20 (brs, 4H), 3.12-2.69 (brs, 4H), 2.00-1.72 (m, 2H); MS (ESI) m/z 458 (M+1).

### Example 13(a)

### 7-Chloro-2-{4-[(1,1-dioxidothiomorpholin-4-yl)methyl]phenyl}-3H-imidazo[4,5-b]pyridine

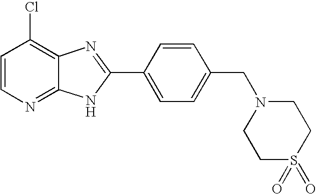

The title compound was prepared in accordance with the general method E using 4-[(1,1-dioxidothiomorpholin-4-yl)methyl]benzoic acid (269 mg, 1.0 mmol), giving 235 mg (62% yield) of the title compound.

MS (ESI) m/z 377 (M+1).

### Example 14

### N-(3-Methoxypropyl)-2-[4-(piperidin-1-ylmethyl)phenyl]-3H-imidazo[4,5-b]pyridine-7-carboxamide hydrochloride

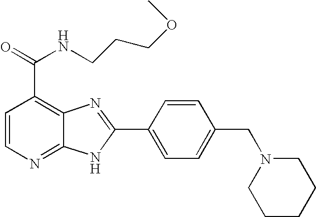

The title compound was prepared in accordance with the general method E using 7-chloro-2-[4-(piperidin-1-ylmethyl)phenyl]-3H-imidazo[4,5-b]pyridine (obtained from Example 14(a)) (120 mg, 0.29 mmol), giving 14 mg (10% yield) of the title compound.

¹H NMR (400 MHz, DMSO-d₆) δ ppm 10.51 (s, 1H), 9.55 (s, 1H), 8.51 (d, 1H), 8.41 (d, 2H), 7.84 (d, 2H), 7.74 (d, 1H), 4.36 (d, 2H), 3.37-3.30 (m, 4H), 3.29 (s, 3H), 2.88 (s, 2H), 1.97-1.83 (m, 2H), 1.83-1.75 (m, 4H), 1.74-1.61 (m, 2H). MS (ESI) m/z 408 (M+1).

### Example 14(a)

### 7-Chloro-2-[4-(piperidin-1-ylmethyl)phenyl]-3H-imidazo[4,5-b]pyridine

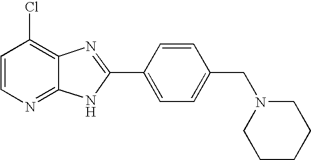

The title compound was prepared in accordance with the general method E using 4-(piperidin-1-ylmethyl)benzoic acid (220 mg, 1.0 mmol), giving 239 mg (73% yield) of the title compound.

MS (ESI) m/z 327 (M+1).

### Example 15

### N-(3-Methoxypropyl)-2-[3-(morpholin-4-ylmethyl)phenyl]-3H-imidazo[4,5-b]pyridine-7-carboxamide hydrochloride

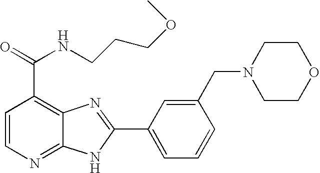

The title compound was prepared in accordance with the general method E using 7-chloro-2-[3-(morpholin-4-ylmethyl)phenyl]-3H-imidazo[4,5-b]pyridine (obtained from Example 15a) (120 mg, 0.28 mmol), giving 19 mg (14% yield) of the title compound.

¹H NMR (400 MHz, MeOH) δ ppm 8.61 (d, 1H), 8.53 (s, 1H), 8.41 (d, 1H), 7.88 (t, 2 H), 7.78 (t, 1H), 4.55 (s, 2H), 4.15-3.94 (m, 2H), 3.94-3.75 (m, 2H), 3.65 (t, 2H), 3.57 (t, 2H), 3.48 (d, 2H), 3.38 (s, 3H), 2.11-1.86 (m, 2H); MS (ESI) m/z 410 (M+1).

### Example 15(a)

### 7-Chloro-2-[3-(morpholin-4-ylmethyl)phenyl]-3H-imidazo[4,5-b]pyridine

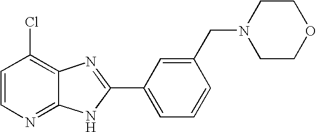

The title compound was prepared in accordance with the general method E using 3-(morpholin-4-ylmethyl)benzoic acid (232 mg, 1.05 mmol), giving 180 mg (55% yield) of the title compound.

MS (APPI) m/z 329 (M+1).

### Example 16

### N-(3-Methoxypropyl)-2-{3-[(4-methylpiperazin-1-yl)methyl]phenyl}-3H-imidazo[4,5-b]pyridine-7-carboxamide hydrochloride

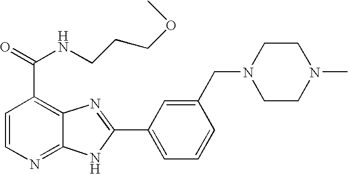

The title compound was prepared in accordance with the general method E using 7-chloro-2-{3-[(4-methylpiperazin-1-yl)methyl]phenyl}-3H-imidazo[4,5-b]pyridine (obtained from Example 16(a)) (100 mg, 0.23 mmol), giving 10 mg (8% yield) of the title compound.

¹H NMR (400 MHz, DMSO-d₆) δ ppm 9.54 (s, 1H), 8.55 (s, 1H), 8.50 (d, 1H), 8.34 (d, 1H), 7.86-7.57 (m, 3H), 3.29 (s, 3H), 2.82 (s, 4H), 2.02-1.76 (m, 2H); MS (ESI) m/z 421 (M+1).

### Example 16(a)

### 7-Chloro-2-{3-[(4-methylpiperazin-1-yl)methyl]phenyl}-3H-imidazo[4,5-b]pyridine

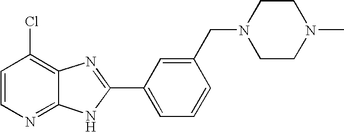

The title compound was prepared in accordance with the general method E using 3-[(4-methylpiperazin-1-yl)methyl]benzoic acid (234 mg, 1.0 mmol), giving 215 mg (63% yield) of the title compound.

MS (ESI) m/z 342 (M+1).

### Example 17

### 2-{4-[(Dipropylamino)sulfonyl]phenyl}-N-(3-methoxypropyl)-3H-imidazo[4,5-b]pyridine-7-carboxamide hydrochloride

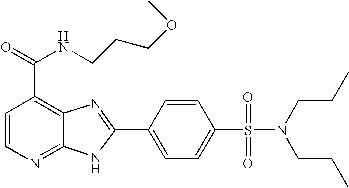

The title compound was prepared in accordance with the general method F using 4-(7-chloro-3H-imidazo[4,5-b]pyridin-2-yl)-N,N-dipropylbenzenesulfonamide (obtained from Example 17(a)) (220 mg, 0.56 mmol), giving 34 mg (12% yield) of the title compound.

¹H NMR (400 MHz, DMSO-d₆) δ ppm 14.31 (s, 1H), 9.53 (s, 1H), 8.70-8.34 (m, 3 H), 8.03 (d, 2H), 7.76 (d, 1H), 3.73-3.38 (m, 4H), 3.28 (s, 3H), 3.21-2.94 (m, 4H), 2.01-1.71 (m, 2H), 1.61-1.29 (m, 4H), 0.83 (t, 6H). MS (ESI) m/z 474 (M+1).

### Example 17(a)

### 4-(7-Chloro-3H-imidazo[4,5-b]pyridin-2-yl)-N,N-dipropylbenzenesulfonamide

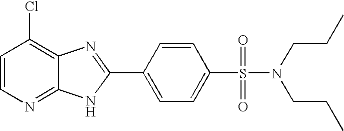

The title compound was prepared in accordance with the general method A using 2,3-diaminopyridine (272 mg, 10.0 mmol) and 4-[(dipropylamino)sulfonyl]benzoic acid (2.85 g, 10.0 mmol) giving 1.83 g (46%); MS (APPI) m/z 393 (M+1).

### Example 18

### N-(3-Methoxypropyl)-2-[4-(methylsulfonyl)phenyl]-3H-imidazo[4,5-b]pyridine-7-carboxamide hydrochloride

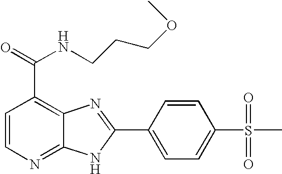

The title compound was prepared in accordance with the general method F using 7-chloro-2-[4-(methylsulfonyl)phenyl]-3H-imidazo[4,5-b]pyridine (obtained from Example 18(a)) (220 mg, 0.56 mmol), giving 16 mg (4% yield) of the title compound.

¹HNMR (400 MHz, DMSO-d₆) δ ppm 9.49 (s, 1H), 8.59 (d, 2H), 8.55 (d, 1H), 8.16 (d, 2H), 7.76 (d, 1H), 3.71-3.46 (m, 4H), 3.34-3.30 (m, 3H), 3.31-3.25 (m, 3H), 1.99-1.74 (m, 2H); MS (AP) m/z 389 (M+1).

### Example 18(a)

### 7-Chloro-2-[4-(methylsulfonyl)phenyl]-3H-imidazo[4,5-b]pyridine

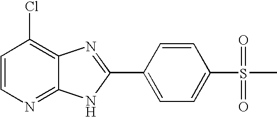

The title compound was prepared in accordance with the general method A using 2,3-diaminopyridine (1.09 g, 10.0 mmol) and 4-(methylsulfone)benzoic acid (2.0 g, 10.0 mmol) giving 568 mg (18%) of the title compound.

MS (APPI) m/z 308 (M+1).

### Example 19

### N-(3-Methoxypropyl)-2-[3-(methylsulfonyl)phenyl]-3H-imidazo[4,5-b]pyridine-7-carboxamide hydrochloride

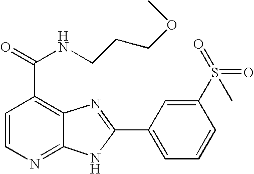

The title compound was prepared in accordance with the general method F using 7-chloro-2-[3-(methylsulfonyl)phenyl]-3H-imidazo[4,5-b]pyridine (obtained from Example 19(a)) (180 mg, 0.58 mmol), giving 25 mg (8.5% yield) of the title compound (triflate salt).

¹H NMR (400 MHz, DMSO-d₆) δ ppm 14.34 (s, 1H), 9.52 (s, 1H), 8.86 (s, 1H), 8.67 (d, 1H), 8.53 (d, 1H), 8.14 (d, 1H), 7.92 (t, 1H), 7.75 (d, 1H), 3.82-3.43 (m, 4H), 3.34 (s, 3H), 3.28 (s, 3H), 2.13-1.51 (m, 2H); ¹⁹F NMR (376 MHz, DMSO-d₆) δ ppm −74.03 (s, 3 F); MS (AP) m/z 389 (M+1).

### Example 19(a)

### 7-Chloro-2-[3-(methylsulfonyl)phenyl]-3H-imidazo[4,5-b]pyridine

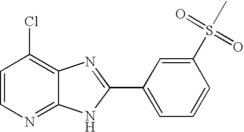

The title compound was prepared in accordance with the general method A using 2,3-diaminopyridine (1.09 g, 10.0 mmol) and 3-(methylsulfone)benzoic acid (2.0 g, 10.0 mmol) giving 921 mg (30%) of the title compound.

MS (APPI) m/z 308 (M+1).

### Example 20

### 2-[4-(morpholin-4-ylmethyl)phenyl]-N-pyridin-3-yl-3H-imidazo[4,5-b]pyridine-7-carboxamide

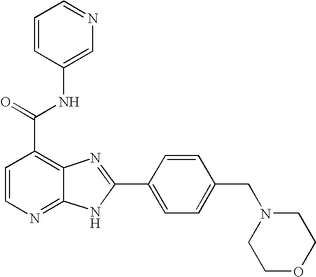

Methyl 8-[4-(morpholin-4-ylmethyl)phenyl]-2,7,9-triazabicyclo[4.3.0]nona-1,3,5,7-tetraene-5-carboxylate (obtained from Example 20(a)) (0.12 mmol) and lithium hydroxide (0.62 mmol) were mixed together in 3 mL THF:water (9:1) and stirred at +120° C. for 10 min in a microwave reactor. The mixture was evaporated, co-evaporated with toluene and dried over vacuum for 4 hours. The crude mixture was divided in two, and one half of the material was mixed with O-benzotriazol-1-yl-N—N—N′,N′-tetramethyluronium hexafluorophosphate (0.15 mmol) and (i-Pr)₂NEt (0.26 mmol) in 2 mL dry DMF. After stirring for 30 min 3-aminopyridine (0.14 mmol) was added and stirring was continued at r.t. over night. The reaction was filtered and purified by preparative HPLC. The fractions containing the product were pooled and extracted with ethyl acetate. The organic layer was dried, filtered and evaporated to yield 4 mg (16%) of the title product.

¹H NMR (400 MHz, DMSO-d₆) δ ppm 9.04 (d, J=1.77 Hz, 1H) 8.54 (d, J=5.05 Hz, 1 H) 8.31-8.45 (m, 4H) 7.80 (d, J=4.80 Hz, 1H) 7.58 (d, J=8.34 Hz, 2H) 7.50 (dd, J=8.21, 4.67 Hz, 1H) 3.60 (s, 6H) 2.35-2.46 (m, 4H). MS (ESI) m/z 413 (M−1).

### Example 20(a)

### Methyl 8-[4-(morpholin-4-ylmethyl)phenyl]-2,7,9-triazabicyclo[4.3.0]nona-1,3,5,7-tetraene-5-carboxylate

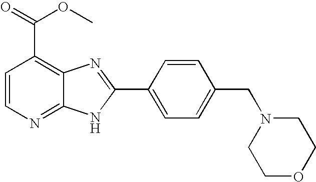

7-Iodo-2-[4-(morpholin-4-ylmethyl)phenyl]-3H-imidazo[4,5-b]pyridine (2.3 mmol, obtained from Example 5(f)) was mixed with 1,3-bis(diphenylphosphino)propane (95 mg, 0.23 mmol), Pd(OAc)₂ (26 mg, 0.11 mmol) and triethylamine (5.75 mmol) in methanol in an autoclave and purged with nitrogen followed by carbon monoxide (g). The vessel was pressurized to 5 bar with carbon monoxide (g) and heated to +100° C. over night. Another 0.12 mmol of 1,3-bis(diphenylphosphino)propane was added and the reaction was continued as above another night. The reaction mixture was allowed to cool to r.t., filtered and the solvent was evaporated in vacuo. The residue was purified by preparative HPLC, which afforded the product as a base, 45 mg (6%) of the title compound.

¹H NMR (400 MHz, DMSO-d₆) δ ppm 8.49 (d, J=4.98 Hz, 1H) 8.29 (d, J=8.20 Hz, 1 H) 7.63 (d, J=4.98 Hz, 1H) 7.52 (d, J=8.20 Hz, 2H) 4.00 (s, 3H) 3.60 (s, 4H) 3.57 (s, 2H) 2.37-2.44 (m, 4H) MS (ESI) m/z 353 (M+1).

### Example 21

### N-Cyclopentyl-8-[4-(morpholin-4-ylmethyl)phenyl]-2,7,9-triazabicyclo[4.3.0]nona-1,3,5,7-tetraene-5-carboxamide

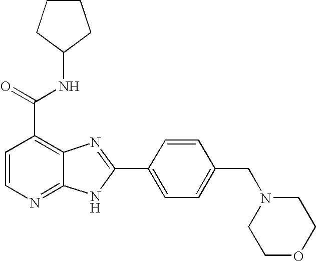

The title compound was synthesized in analogy with Example 20 using half of the 8-[4-(morpholin-4-ylmethyl)phenyl]-2,7,9-triazabicyclo[4.3.0]nona-1,3,5,7-tetraene-5-carboxylic acid prepared in Example 20, and using cyclopentylamine (0.13 mmol) instead of 3-aminopyridine. The reaction yielded 4 mg (16%) of the product.

¹H NMR (400 MHz, Chloroform-d) δ ppm 9.84 (d, J=7.33 Hz, 1H) 8.56 (d, J=5.05 Hz, 1H) 8.23 (d, J=8.34 Hz, 2H) 8.09 (d, J=5.05 Hz, 1H) 7.65 (d, J=8.08 Hz, 2H) 4.54-4.63 (m, 1H) 3.73-3.81 (m, 4H) 3.66 (s, 2H) 2.48-2.58 (m, 4H) 2.12-2.22 (m, 2 H) 1.85-1.96 (m, 2H) 1.73-1.83 (m, 4H). MS (ESI) m/z 404 (M−1).

### Example 22

### N-(3-Methoxybenzyl)-2-[4-(morpholin-4-ylmethyl)phenyl]-3H-imidazo[4,5-b]pyridine-7-carboxamide

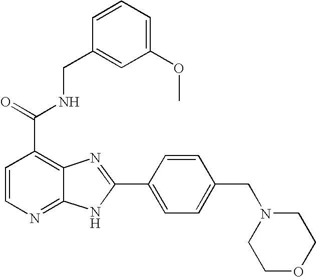

Triethylamine (45 mg, 0.44 mmol), TSTU (56 mg, 0.18 mmol) and 8-[4-(morpholin-4-ylmethyl)phenyl]-2,7,9-triazabicyclo[4.3.0]nona-1,3,5,7-tetraene-5-carboxylic acid (50 mg, 0.15 mmol, obtained from Example 20) was dissolved in DMF (2 mL) and stirred at r.t. for 15 minutes. 1-(3-methoxyphenyl)methanamine (26 mg, 0.19 mmol) was added and the mixture stirred for 3 h. The crude product was purified by preparative HPLC affording 35 mg (51%).

¹H NMR (DMSO-d₆) δ ppm 14.07 (br s, 1H), 10.01 (br s, 1H), 8.52-8.45 (m, 1H), 8.31-8.21 (m, 2H), 7.77-7.72 (m, 1H), 7.57-7.48 (m, 2H), 7.35-7.28 (m, 1H), 7.10-7.00 (m, 2H), 6.92-6.85 (m, 1H), 4.75-4.66 (m, 2H), 3.75 (s, 3H), 3.65-3.51 (m, 6H), 2.45-2.35 (m, 4H); MS (ESI) m/z 458 (M+1).

### Example 23

### 3-[4-({2-[4-(Morpholin-4-ylmethyl)phenyl]-3H-imidazo[4,5-b]pyridin-7-yl}carbonyl)piperazin-1-yl]propanenitrile

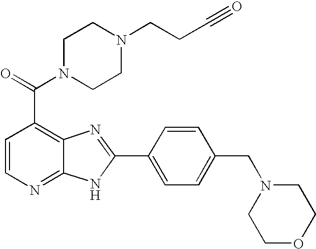

Triethylamine (36 mg, 0.35 mmol), TSTU (44 mg, 0.15 mmol) and 8-[4-(morpholin-4-ylmethyl)phenyl]-2,7,9-triazabicyclo[4.3.0]nona-1,3,5,7-tetraene-5-carboxylic acid (40 mg, 0.12 mmol, obtained from Example 20) was dissolved in DMF (2 mL) and stirred at r.t. for 15 minutes. 3-piperazin-1-ylpropanenitrile (21 mg, 0.15 mmol) was added and the mixture stirred for 1.5 h. The crude product was purified by preparative HPLC affording 28 mg (51%).

¹HNMR (DMSO-d₆) δ ppm 13.76 (br s, 1H), 8.36 (d, 1H), 8.23-8.18 (m, 2H), 7.53-7.48 (m, 2H), 7.21 (d, 1H), 3.81-3.70 (m, 2H), 3.64-3.52 (m, 6H), 3.30-3.18 (m, 2 H), 2.73-2.56 (m, 6H), 2.47-2.31 (m, 6H); MS (ESI) m/z 460 (M+1).

### Pharmaceutical Compositions

According to one aspect of the present invention there is provided a pharmaceutical composition comprising a compound of formula I, as a free base or a pharmaceutically acceptable salt, solvate or solvate of salt thereof, for use in the prevention and/or treatment of conditions associated with glycogen synthase kinase-3.

The composition may be in a form suitable for oral administration, for example as a tablet, for parenteral injection as a sterile solution or suspension. In general the above compositions may be prepared in a conventional manner using pharmaceutically carriers or diluents. Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

A compound of formula I, or a pharmaceutically acceptable salt, solvate or solvate of salt thereof, can be used on its own but will usually be administered in the form of a pharmaceutical composition in which the formula I compound/salt/solvate (active ingredient) is in association with a pharmaceutically acceptable excipient, diluent or carrier. Dependent on the mode of administration, the pharmaceutical composition may comprise from 0.05 to 99% w (percent by weight), for example from 0.10 to 50% w, of active ingredient, all percentages by weight being based on total composition.

An excipient, diluent or carrier includes water, aqueous polyethylene glycol, magnesium carbonate, magnesium stearate, talc, a sugar (such as lactose), pectin, dextrin, starch, tragacanth, microcrystalline cellulose, methyl cellulose, sodium carboxymethyl cellulose or cocoa butter.

A composition of the invention can be in tablet or injectable form. The tablet may additionally comprise a disintegrant and/or may be coated (for example with an enteric coating or coated with a coating agent such as hydroxypropyl methylcellulose).

The invention further provides a process for the preparation of a pharmaceutical composition of the invention which comprises mixing a compound of formula I, or a pharmaceutically acceptable salt, solvate or solvate of salt thereof, as hereinbefore defined, with a pharmaceutically acceptable excipient, diluent or carrier.

An example of a pharmaceutical composition of the invention is an injectable solution containing a compound of the invention, or a pharmaceutically acceptable salt, solvate or solvate of salt thereof, as hereinbefore defined, and sterile water, and, if necessary, either sodium hydroxide or hydrochloric acid to bring the pH of the final composition to about pH 5, and optionally a surfactant to aid dissolution.

### Medical Use

Surprisingly, it has been found that the compounds defined in the present invention, as a free base or a pharmaceutically acceptable salt thereof, are well suited for inhibiting glycogen synthase kinase-3 (GSK3). Accordingly, the compounds of the present invention are expected to be useful in the prevention and/or treatment of conditions associated with glycogen synthase kinase-3 activity, i.e. the compounds may be used to produce an inhibitory effect of GSK3 in mammals, including man, in need of such prevention and/or treatment.

GSK3 is highly expressed in the central and peripheral nervous system and in other tissues. Thus, it is expected that compounds of the invention are well suited for the prevention and/or treatment of conditions associated with glycogen synthase kinase-3 in the central and peripheral nervous system. In particular, the compounds of the invention are expected to be suitable for prevention and/or treatment of conditions associated with especially, dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica.

Other conditions are selected from the group consisting of amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication.

Further conditions are selected from the group consisting of predemented states, Mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia and Type I and Type II diabetes, diabetic neuropathy and diabetes related disorders.

One embodiment of the invention relates to the prevention and/or treatment of dementia and Alzheimer's Disease.

Another embodiment of the invention relates to the prevention and/or treatment of bone-related disorders.

The dose required for the therapeutic or preventive treatment of a particular disease will necessarily be varied depending on the host treated, the route of administration and the severity of the illness being treated.

The present invention relates also to the use of a compound of formula I as defined hereinbefore, in the manufacture of a medicament for the prevention and/or treatment of conditions associated with glycogen synthase kinase-3.

In the context of the present specification, the term “therapy” also includes “prevention” unless there are specific indications to the contrary. The terms “therapeutic” and “therapeutically” should be construed accordingly.

The invention also provides for a method of treatment and/or prevention of conditions associated with glycogen synthase kinase-3 comprising administering to a mammal, including man in need of such treatment and/or prevention a therapeutically effective amount of a compound of formula I, as hereinbefore defined.

### Non-Medical Use

In addition to their use in therapeutic medicine, the compounds of formula I as a free base or a pharmaceutically acceptable salt thereof, are also useful as pharmacological tools in the development and standardisation of in vitro and in vivo test systems for the evaluation of the effects of inhibitors of GSK3 related activity in laboratory animals such as cats, dogs, rabbits, monkeys, rats and mice, as part of the search for new therapeutics agents.

### Pharmacology

### Determination of ATP Competition in Scintillation Proximity GSK3β Assay.

### GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK30 (Dundee University, UK), 12 mM morpholinepropanesulfonic acid (MOPS), pH 7.0, 0.3 mM EDTA, 0.01% β-mercaptoethanol, 0.004% Brij 35 (a natural detergent), 0.5% glycerol and 0.5 μg BSA/25 μl. The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3, used to calculate the inhibition constants (Kᵢ) of the various compounds, was 20 μM.

The following abbreviations have been used:

MOPS Morpholinepropanesulfonic acid
EDTA Ethylenediaminetetraacetic acid

### BSA Bovin Serum Albumin

### ATP Adenosine Triphosphate

### SPA Scintillation Proximity Assay

GSK3 Glycogen synthase kinase 3

### Results

Typical Kᵢ values for the compounds of the present invention are in the range of about 0.001 to about 10,000 nM. Other values for Kᵢ are in the range of about 0.001 to about 1000 nM. Further values for Kᵢ are in the range of about 0.001 nM to about 300 nM.

TABLE 1
Specimen results from assay.

| Example no | Kᵢ (nM) | n |
|---|---|---|
| 3 | 17 | 4 |
| 4 | 8 | 2 |
| 6 | 51 | 3 |
| 7 | 36 | 3 |

1. A compound of formula I: 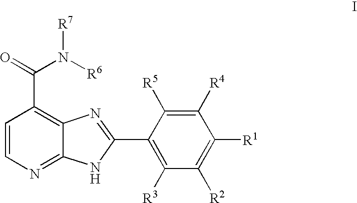
wherein;
R¹ is selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ are independently selected from hydrogen, C₁₋₃alkyl and C₁₋₃haloalkyl;
R⁶ and R⁷ are independently selected from hydrogen, C₁₋₆alkyl, C₁₋₆alkylaryl, aryl and heteroaryl, said C₁₋₆alkyl, C₁₋₆alkylaryl, aryl and heteroaryl are optionally substituted with one or more A; or R⁶ and R⁷ may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more R^{j} or A;
R^{a} is hydrogen, C₁₋₃alkyl or C₁₋₃haloalkyl, said C₁₋₃alkyl or C₁₋₃haloalkyl is optionally substituted with one or more C₁₋₃alkoxy;
R^{b} and R^{c} are independently selected from hydrogen, C₁₋₆alkyl and C₁₋₆haloalkyl, said C₁₋₆alkyl or C₁₋₆haloalkyl is optionally substituted with one or more OR^{a} or NR^{d}R^{e} or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃alkoxy and in which any sulphur atom is optionally oxidised to —SO₂—;
R^{d} and R^{e} are independently selected from hydrogen, C₁₋₆alkyl and C₁₋₆haloalkyl, said C₁₋₆alkyl or C₁₋₆haloalkyl is optionally substituted with one or more OR^{a}; or R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is hydrogen, C₁₋₃alkyl or C₁₋₃haloalkyl, said C₁₋₃alkyl or C₁₋₃haloalkyl is optionally substituted with one or more C₁₋₃alkoxy;
Rⁱ is C₁₋₃alkyl or C₁₋₃haloalkyl, said C₁₋₃alkyl or C₁₋₃haloalkyl is optionally substituted with one or more OR^{a};
R^{j} is aryl or heteroaryl, wherein said aryl or heteroaryl is optionally substituted with one or more C₁₋₃alkyl, OR^{a}, halo or CN;
A is halo, CN, OR^{a}, or NR^{b}R^{c};
as a base or a pharmaceutically acceptable salt, solvate or solvate of a salt thereof.

2. A compound of formula I: 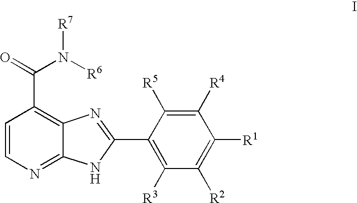
wherein;
R¹ is hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ or C(O)R^{j};
R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ are independently selected from hydrogen, C₁₋₃alkyl and C₁₋₃haloalkyl;
R⁶ and R⁷ are independently selected from hydrogen, C₁₋₆alkyl and heteroaryl, said C₁₋₆alkyl and heteroaryl optionally substituted with one or more A;
R^{a} is hydrogen, C₁₋₃alkyl or C₁₋₃haloalkyl, said C₁₋₃alkyl or C₁₋₃haloalkyl optionally substituted with one or more C₁₋₃alkoxy;
R^{b} and R^{c} are independently selected from hydrogen, C₁₋₆alkyl and C₁₋₆haloalkyl, said C₁₋₆alkyl or C₁₋₆haloalkyl optionally substituted with one or more OR^{a} or NR^{d}R^{e} or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{d} and R^{e} are independently selected from hydrogen, C₁₋₆alkyl and C₁₋₆haloalkyl, said C₁₋₆alkyl or C₁₋₆haloalkyl optionally substituted with one or more OR^{a}; or R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is hydrogen, C₁₋₃alkyl or C₁₋₃haloalkyl, said C₁₋₃alkyl or C₁₋₃haloalkyl optionally substituted with one or more C₁₋₃alkoxy;
Rⁱ is C₁₋₃alkyl or C₁₋₃haloalkyl, said C₁₋₃alkyl or C₁₋₃haloalkyl optionally substituted with one or more OR^{a};
R^{j} is aryl or heteroaryl, wherein said aryl or heteroaryl is optionally substituted with one or more C₁₋₃alkyl, OR^{a}, halo or CN;
A is halo, CN, OR^{a}, or NR^{b}R^{c};
as a base or a pharmaceutically acceptable salt, solvate or solvate of a salt thereof.

3. A compound according to claim 1, wherein
R¹ is hydrogen, C₁₋₃haloalkyl, SO₂NR^{b}R^{c}, C(O)NR^{b}R^{c}, CH₂NR^{b}R^{c}, CH₂OR^{h}, or SO₂Rⁱ;
R² and R⁴ are independently selected from hydrogen, halo, CN, NO₂, C₁₋₃alkyl, C₁₋₃haloalkyl, OR^{a}, C(O)NR^{b}R^{c}, CH₂NR^{b}R^{c}, CH₂OR^{h}, and SO₂Rⁱ;
R³ and R⁵ are independently selected from hydrogen and C₁₋₃haloalkyl;
R⁶ and R⁷ are independently selected from hydrogen, C₁₋₆alkyl and heteroaryl, said C₁₋₆alkyl and heteroaryl optionally substituted with one or more A;
R^{a} is C₁₋₃alkyl or C₁₋₃haloalkyl;
R^{b} and R^{c} are independently selected from C₁₋₆alkyl and C₁₋₆haloalkyl; or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is C₁₋₃alkyl or C₁₋₃haloalkyl;
Rⁱ is C₁₋₃alkyl or C₁₋₃haloalkyl;
A is halo, CN, OR³, or NR^{b}R^{c};
as a base or a pharmaceutically acceptable salt, solvate or solvate of a salt thereof.

4. A compound according to claim 1, wherein
R¹ is hydrogen, C₁₋₃haloalkyl, SO₂NR^{b}R^{c}, C(O)NR^{b}R^{c}, CH₂NR^{c}R^{c}, or SO₂Rⁱ;
R² and R⁴ are independently selected from hydrogen, C₁₋₃haloalkyl, CH₂OR^{h}, and SO₂Rⁱ;
R³ and R⁵ are independently selected from hydrogen and C₁₋₃haloalkyl;
R⁶ and R⁷ are independently selected from hydrogen and C₁₋₆alkyl, said C₁₋₆alkyl optionally substituted with one or more A;
R^{a} is C₁₋₃alkyl;
R^{b} and R^{c} are independently C₁₋₆alkyl; or R^{b} and R^{c} may, together with the atom to which they are attached, form a 6-membered heterocyclic ring containing one or more heteroatoms selected from N or O, wherein said heterocyclic ring is optionally substituted with one C₁₋₃alkyl;
R^{h} is C₁₋₃haloalkyl;
Rⁱ is C₁₋₃alkyl;
A is OR^{a};
as a base or a pharmaceutically acceptable salt, solvate or solvate of a salt thereof.

5. A compound according to claim 1, wherein
R¹ is selected from hydrogen, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R² and R⁴ are independently selected from hydrogen, halo, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ are independently selected from hydrogen, C₁₋₃alkyl and C₁₋₃haloalkyl;
R⁶ and R⁷ are independently selected from hydrogen, C₁₋₆alkyl, C₁₋₆alkylaryl, aryl and heteroaryl, said C₁₋₆alkyl, C₁₋₆alkylaryl, aryl and heteroaryl are optionally substituted with one or more A; or R⁶ and R⁷ may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more R^{j} or A;
R^{a} is hydrogen, C₁₋₃alkyl or C₁₋₃haloalkyl, said C₁₋₃alkyl or C₁₋₃haloalkyl is optionally substituted with one or more C₁₋₃alkoxy;
R^{b} and R^{c} are independently selected from hydrogen, C₁₋₆alkyl and C₁₋₆haloalkyl, said C₁₋₆alkyl or C₁₋₆haloalkyl is optionally substituted with one or more OR^{a} or NR^{d}R^{e} or
R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃alkoxy and in which any sulphur atom is optionally oxidised to —SO₂—;
R^{d} and R^{e} are independently selected from hydrogen, C₁₋₆alkyl and C₁₋₆haloalkyl, said C₁₋₆alkyl or C₁₋₆haloalkyl is optionally substituted with one or more OR^{a}; or
R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is hydrogen, C₁₋₃alkyl or C₁₋₃haloalkyl, said C₁₋₃alkyl or C₁₋₃haloalkyl is optionally substituted with one or more C₁₋₃alkoxy;
Rⁱ is C₁₋₃alkyl or C₁₋₃haloalkyl, said C₁₋₃alkyl or C₁₋₃haloalkyl is optionally substituted with one or more OR^{a};
R^{j} is aryl or heteroaryl, wherein said aryl or heteroaryl is optionally substituted with one or more C₁₋₃alkyl, OR^{a}, halo or CN;
A is halo, CN, OR^{a}, or NR^{b}R^{c};
as a base or a pharmaceutically acceptable salt, solvate or solvate of a salt thereof.

6. A compound according to claim 1, wherein
R¹ is selected from hydrogen, C₁₋₃haloalkyl, C(O)NR^{b}R^{c}, CH₂NR^{b}R^{c}, SO₂Rⁱ and SO₂R^{b}R^{c};
R² and R⁴ are independently selected from hydrogen, halo, C₁₋₃haloalkyl, OR^{a}, CH₂NR^{b}R^{c}, CH₂OR^{h} and SO₂Rⁱ;
R³ and R⁵ are independently selected from hydrogen or C₁₋₃haloalkyl;
R⁶ and R⁷ are independently selected from hydrogen, C₁₋₆alkyl, C₁₋₆alkylaryl, aryl and heteroaryl, said C₁₋₆alkyl, C₁₋₆alkylaryl, aryl and heteroaryl are optionally substituted with one or more A; or R⁶ and R⁷ may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more R^{j} or A;
R^{a} is C₁₋₃alkyl;
R^{b} and R^{c} are independently selected from hydrogen, C₁₋₆alkyl and C₁₋₆haloalkyl or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃alkoxy and in which any sulphur atom is optionally oxidised to —SO₂—;
R^{h} is C₁₋₃haloalkyl,
Rⁱ is C₁₋₃alkyl;
R^{j} is aryl or heteroaryl, wherein said aryl or heteroaryl is optionally substituted with one or more C₁₋₃alkyl, OR^{a}, halo or CN;
A is halo, CN or OR^{a},
as a base or a pharmaceutically acceptable salt, solvate or solvate of a salt thereof.

7. A compound according to claim 1 or claim 2, wherein R² and R³ are hydrogen.

8. A compound according to claim 5, wherein
R¹ is selected from hydrogen, C₁₋₃haloalkyl, C(O)NR^{b}R^{c}, CH₂NR^{b}R^{c}, SO₂Rⁱ and SO₂R^{b}R^{c};
R² and R⁴ are independently selected from hydrogen, halo, C₁₋₃haloalkyl, OR^{a}, CH₂NR^{b}R^{c}, CH₂OR^{h} and SO₂Rⁱ;
R⁶ and R⁷ are independently selected from hydrogen, C₁₋₆alkyl, C₁₋₆alkylaryl, aryl and heteroaryl, said C₁₋₆alkyl, C₁₋₆alkylaryl, aryl and heteroaryl are optionally substituted with one or more A; or R⁶ and R⁷ may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more R^{j} or A;
R^{a} is C₁₋₃alkyl;
R^{b} and R^{c} are independently selected from hydrogen, C₁₋₆alkyl and C₁₋₆haloalkyl or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃alkoxy and in which any sulphur atom is optionally oxidised to —SO₂—;
R^{h} is C₁₋₃haloalkyl;
Rⁱ is C₁₋₃alkyl;
R^{j} is aryl or heteroaryl, wherein said aryl or heteroaryl is optionally substituted with one or more C₁₋₃alkyl, OR^{a}, halo or CN;
A is halo, CN or OR^{a},
as a base or a pharmaceutically acceptable salt, solvate or solvate of a salt thereof.

9. A compound according to claim 8, wherein
R⁶ and R⁷ are independently selected from hydrogen and C₁₋₆alkyl, said C₁₋₆alkyl is substituted with one OR^{a} and R^{a} is C₁₋₃alkyl.

10. A compound according to claim 9, wherein said C₁₋₆alkyl is propyl and R^{a} is methyl.

11. A compound according to claim 8, wherein said C₁₋₆alkylalkyl in R⁶ or R⁷ is C₁₋₃alkylaryl.

12. A compound according to claim 1, selected from:
N-(3-Methoxypropyl)-2-[2-(trifluoromethyl)phenyl]-3H-imidazo[4,5-b]pyridine-7-carboxamide hydrochloride;
N-(3-Methoxypropyl)-2-[3-(trifluoromethyl)phenyl]-3H-imidazo[4,5-b]pyridine-7-carboxamide hydrochloride;
N-(3-Methoxypropyl)-2-[4-(trifluoromethyl)phenyl]-3H-imidazo[4,5-b]pyridine-7-carboxamide hydrochloride;
N-(3-Methoxypropyl)-2-{3-[(2,2,3,3-tetrafluoropropoxy)methyl]phenyl}-3H-imidazo[4,5-b]pyridine-7-carboxamide hydrochloride;
N-(3-Methoxypropyl)-2-[4-(morpholin-4-ylmethyl)phenyl]-3H-imidazo[4,5-b]pyridine-7-carboxamide hydrochloride;
N-(3-Methoxypropyl)-2-{4-[(4-methylpiperazin-1-yl)carbonyl]phenyl}-3H-imidazo[4,5-b]pyridine-7-carboxamide hydrochloride;
N-(3-Methoxypropyl)-2-[4-(morpholin-4-ylcarbonyl)phenyl]-3H-imidazo[4,5-b]pyridine-7-carboxamide hydrochloride;
2-[3-Fluoro-4-(morpholin-4-ylmethyl)phenyl]-N-(3-methoxypropyl)-3H-imidazo[4,5-b]pyridine-7-carboxamide hydrochloride;
2-[3-Methoxy-4-(morpholin-4-ylmethyl)phenyl]-N-(3-methoxypropyl)-3H-imidazo[4,5-b]pyridine-7-carboxamide hydrochloride;
N-(3-Methoxypropyl)-2-[4-(morpholin-4-ylmethyl)-3-(trifluoromethyl)phenyl]-3H-imidazo[4,5-b]pyridine-7-carboxamide hydrochloride;
N-(3-Methoxypropyl)-2-[4-(pyrrolidin-1-ylsulfonyl)phenyl]-3H-imidazo[4,5-b]pyridine-7-carboxamide hydrochloride;
N-(3-Methoxypropyl)-2-{4-[(4-methylpiperazin-1-yl)sulfonyl]phenyl}-3H-imidazo[4,5-b]pyridine-7-carboxamide hydrochloride;
2-{4-[(1,1-Dioxidothiomorpholin-4-yl)methyl]phenyl}-N-(3-methoxypropyl)-3H-imidazo[4,5-b]pyridine-7-carboxamide hydrochloride;
N-(3-Methoxypropyl)-2-[4-(piperidin-1-ylmethyl)phenyl]-3H-imidazo[4,5-b]pyridine-7-carboxamide hydrochloride;
N-(3-Methoxypropyl)-2-[3-(morpholin-4-ylmethyl)phenyl]-3H-imidazo[4,5-b]pyridine-7-carboxamide hydrochloride;
N-(3-Methoxypropyl)-2-{3-[(4-methylpiperazin-1-yl)methyl]phenyl}-3H-imidazo[4,5-b]pyridine-7-carboxamide hydrochloride;
2-{4-[(Dipropylamino)sulfonyl]phenyl}-N-(3-methoxypropyl)-3H-imidazo[4,5-b]pyridine-7-carboxamide hydrochloride;
N-(3-Methoxypropyl)-2-[4-(methylsulfonyl)phenyl]-3H-imidazo[4,5-b]pyridine-7-carboxamide hydrochloride;
N-(3-Methoxypropyl)-2-[3-(methylsulfonyl)phenyl]-3H-imidazo[4,5-b]pyridine-7-carboxamide hydrochloride;
2-[4-(Morpholin-4-ylmethyl)phenyl]-N-pyridin-3-yl-3H-imidazo[4,5-b]pyridine-7-carboxamide;
N-Cyclopentyl-8-[4-(morpholin-4-ylmethyl)phenyl]-2,7,9-triazabicyclo[4.3.0]nona-1,3,5,7-tetraene-5-carboxamide;
N-(3-Methoxybenzyl)-2-[4-(morpholin-4-ylmethyl)phenyl]-3H-imidazo[4,5-b]pyridine-7-carboxamide; and
3-[4-({2-[4-(Morpholin-4-ylmethyl)phenyl]-3H-imidazo[4,5-b]pyridin-7-yl}carbonyl)piperazin-1-yl]propanenitrile;
as a base or a pharmaceutically acceptable salt, solvate or solvate of a salt thereof.

13. A pharmaceutical formulation comprising as active ingredient a therapeutically effective amount of a compound according to claim 1 in association with pharmaceutically acceptable excipients, carriers or diluents.

14-19. (canceled)

20. A method of prevention and/or treatment of dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica, comprising administering to a mammal, including man in need of such prevention and/or treatment, a therapeutically effective amount of a compound of formula I as defined in claim 1.

21. A method according to claim 20, wherein the disease is Alzheimer's Disease.

22. A method of prevention and/or treatment of amyotrophic lateral sclerosis, corticobasal degeneration, Down syndrome, Huntington's Disease, postencephalitic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication, comprising administering to a mammal, including man in need of such prevention and/or treatment, a therapeutically effective amount of a compound of formula I as defined in claim 1.

23. A method of prevention and/or treatment of predemented states, Mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia and Type I and Type II diabetes, diabetic neuropathy and diabetes related disorders, comprising administering to a mammal, including man in need of such prevention and/or treatment, a therapeutically effective amount of a compound of formula I as defined in claim 1.

24. A method of prevention and/or treatment of bone-related disorders, comprising administering to a mammal, including man in need of such prevention and/or treatment, a therapeutically effective amount of a compound of formula I as defined in claim 1.

25. (canceled)

26. A compound selected from:
7-Chloro-2-[2-(trifluoromethyl)phenyl]-3H-imidazo[4,5-b]pyridine;
7-Chloro-2-[3-(trifluoromethyl)phenyl]-3H-imidazo[4,5-b]pyridine;
7-Chloro-2-[4-(trifluoromethyl)phenyl]-3H-imidazo[4,5-b]pyridine;
7-Chloro-2-{3-[(2,2,3,3-tetrafluoropropoxy)methyl]phenyl}-3H-imidazo[4,5-b]pyridine;
Methyl 4-(3H-imidazo[4,5-b]pyridin-2-yl)benzoate;
7-Iodo-2-[4-(morpholin-4-ylmethyl)phenyl]-3H-imidazo[4,5-b]pyridine;
Methyl 4-(7-chloro-3H-imidazo[4,5-b]pyridin-2-yl)benzoate;
4-(7-Chloro-3H-imidazo[4,5-b]pyridin-2-yl)benzoic acid;
7-Chloro-2-[4-(morpholin-4-ylcarbonyl)phenyl]-3H-imidazo[4,5-b]pyridine;
7-Chloro-2-[4-(morpholin-4-ylmethyl)phenyl]-3H-imidazo[4,5-b]pyridine;
7-Chloro-2-[3-fluoro-4-(morpholin-4-ylmethyl)phenyl]-3H-imidazo[4,5-b]pyridine;
7-Chloro-2-[3-methoxy-4-(morpholin-4-ylmethyl)phenyl]-3H-imidazo[4,5-b]pyridine;
7-Chloro-2-[4-(morpholin-4-ylmethyl)-3-(trifluoromethyl)phenyl]-3H-imidazo[4,5-b]pyridine;
7-Chloro-2-[4-(pyrrolidin-1-ylsulfonyl)phenyl]-3H-imidazo[4,5-b]pyridine;
7-Chloro-2-{4-[(4-methylpiperazin-1-yl)sulfonyl]phenyl}-3H-imidazo[4,5-b]pyridine;
7-Chloro-2-{4-[(1,1-dioxidothiomorpholin-4-yl)methyl]phenyl}-3H-imidazo[4,5-b]pyridine;
7-Chloro-2-[4-(piperidin-1-ylmethyl)phenyl]-3H-imidazo[4,5-b]pyridine;
7-Chloro-2-[3-(morpholin-4-ylmethyl)phenyl]-3H-imidazo[4,5-b]pyridine;
7-Chloro-2-{3-[(4-methylpiperazin-1-yl)methyl]phenyl}-3H-imidazo[4,5-b]pyridine;
4-(7-Chloro-3H-imidazo[4,5-b]pyridin-2-yl)-N,N-dipropylbenzenesulfonamide;
7-Chloro-2-[4-(methylsulfonyl)phenyl]-3H-imidazo[4,5-b]pyridine;
7-Chloro-2-[3-(methylsulfonyl)phenyl]-3H-imidazo[4,5-b]pyridine; or
Methyl 8-[4-(morpholin-4-ylmethyl)phenyl]-2,7,9-triazabicyclo[4.3.0]nona-1,3,5,7-tetraene-5-carboxylate.

27. (canceled)
